(19) United States
(12) Patent Application Publication
(10) Pub. No.: US 2006/0211606 A1
(43) Pub. Date: Sep. 21, 2006

(54) Peptides

(76) Inventors: Geoffrey Goldspink, Harpenden (GB); Shi Yu Yang, London (GB); Paul Goldspink, Chicago, IL (US)

Correspondence Address: NIXON & VANDERHYE, PC, 901 NORTH GLEBE ROAD, 11TH FLOOR, ARLINGTON, VA, 22203, US

(21) Appl. No.: 11/378,624

(22) Filed: Mar. 20, 2006

Related U.S. Application Data
(60) Provisional application No. 60/662,802, filed on Mar. 18, 2005.

(30) Foreign Application Priority Data
Mar. 3, 2006 (WO) PCT/GB06/00773

(51) Int. Cl. A61K 38/30

(52) U.S. Cl. 514/9; 514/12; 530/324; 530/317

(57) ABSTRACT

This invention relates to biologically active polypeptides derived from the E peptide that forms the C-terminus of the insulin-like growth factor I (IGF-I) splice variant known as mechano growth factor (MGF). These peptides are modified to improve their stability compared to the naturally occurring E peptide.

### FIELD OF THE INVENTION

This invention relates to biologically active polypeptides derived from the E domain that forms the C-terminus of the insulin-like growth factor I (IGF-I) splice variant known as mechano growth factor (MGF). These peptides are modified to improve their stability compared to the naturally occurring E domain peptide.

### BACKGROUND TO THE INVENTION

Mammalian IGF-I polypeptides have a number of isoforms, which arise as a result of alternative mRNA splicing. Broadly, there are two types of isoform, liver-type isoforms and non-liver-type ones. Liver-type isoforms may be expressed in the liver or elsewhere but, if expressed elsewhere, are equivalent to those expressed in the liver. They have a systemic action and are the main isoforms in mammals. Non-liver-type isoforms are less common and some are believed to have an autocrine/paracrine action. The MGF isoform to which this invention relates is of the latter type.

In MGF (Yang et al, 1996; McKoy et al, 1999), alternative splicing introduces an insert which changes the reading frame of the C-terminal portion of the molecule. This insert is 49 base pairs long in human MGF. A 52 base pair insert has a similar effect in rat and rabbit MGF. The result is that MGF is slightly longer than liver-type IGF-I (because the terminator codon appears later owing to the reading frame shift) and that the C-terminal E domain has a different sequence. It is also smaller overall because it lacks glycosylation.

In human MGF, the C-terminus is formed by a 24 amino acid E domain, sometimes termed an Ec peptide (SEQ ID NO: 27). In rat and rabbit MGF, the corresponding E domains, sometimes termed Eb peptides, are 25 amino acids in length (SEQ ID NOS: 13/14). Liver-type IGF-I instead contains an Ea peptide at the C-terminus. The sequences of the Ea and Ec/Eb peptides are unrelated to one another because of the reading frame shift discussed above. The presence of a splice variant with what can now be seen to be the MGF C-terminal was first noted by Chew et al (1995), who identified it in liver tissue during studies on patients suffering from liver cancer, but did not investigate it at all in terms of potential function or therapeutic significance.

Goldspink and co-workers have already identified MGF for use against disorders of skeletal muscle, notably muscular dystrophy; for use against disorders of cardiac muscle, notably in the prevention or limitation of myocardial damage in response to ischemia or mechanical overload of the heart; for the treatment of neurological disorders in general; and for nerve repair in particular (WO97/33997; WO01/136483; WO01/85781; WO03/066082). It is becoming increasingly clear that liver-type IGF-I and MGF have different roles and functions. Thus, Hill and Goldspink (2003) have shown that, in the rat anterior tibialis muscle, MGF is expressed rapidly in response to mechanical damage caused by electrical stimulation or resulting from bupivacaine injection, but that its expression then declines within a few days. Conversely, liver-type IGF-I is more slowly upregulated and its increase is commensurate with the decline in MGF expression. In addition, Yang and Goldspink (2002) have shown, using the mouse C2C12 muscle cell line as an in vitro model, that a 24 amino acid peptide related to the Ec peptide from the C-terminus of human MGF, but with Histidine in the penultimate position rather than the native Arginine, and an additional C-terminal cysteine, has a distinct activity compared to that of mature IGF-I in that it increases myoblast proliferation but inhibits myotube formation. Dluzniewska et al (September 2005) have also demonstrated a strong neuroprotective effect of the a related peptide, again with with Histidine in the penultimate position rather than the native Arginine and some modifications by way of conversion of L-Arginine to D-Arginine at positions 14 and 15, plus C-terminal amidation and PEGylation.

### SUMMARY OF THE INVENTION

However, the present inventors have found that the native human MGF C terminal Ec peptide has a short half-life in human plasma. Hence, stabilising modifications can enhance its potential for use as a pharmaceutical.

The inventors have also demonstrated that stabilised MGF C-terminal E peptides have neuroprotective and cardioprotective properties, as well as the ability to increase the strength of normal and dystrophic skeletal muscle.

Accordingly, the invention provides a polypeptide comprising up to 50 amino acid residues;

said polypeptide comprising a sequence of amino acids derived from the C-terminal E peptide of a Mechano Growth Factor (MGF) isoform of Insulin-like Growth Factor I (IGF-I);

said polypeptide incorporating one or more modifications that give it increased stability compared to the unmodified MGF E peptide;

and said polypeptide possessing biological activity.

The invention also provides an extended polypeptide comprising a polypeptide of the invention, extended by non-wild-type amino acid sequence N-terminal and/or C-terminal to said polypeptide.

The invention also provides a composition comprising a polypeptide or extended polypeptide of the invention and a carrier.

The invention also provides a pharmaceutical composition comprising a polypeptide or extended polypeptide of the invention and a pharmaceutically acceptable carrier.

The invention also provides a polypeptide or extended polypeptide of the invention for use in a method of treatment of the human or animal body.

The invention also provides a method of treating a muscular disorder by administering to a patient in need thereof an effective amount of a polypeptide or extended polypeptide of the invention. Said muscular disorder may be, for example, a disorder of skeletal muscle or a disorder of cardiac muscle.

The invention also provides a method of treating a neurological disorder by administering to a patient in need thereof an effective amount of a polypeptide or extended polypeptide of the invention.

The invention also provides use of a polypeptide or extended polypeptide of the invention in the manufacture of a medicament for use in a treatment as defined above.

The invention also provides a method of treating a neurological disorder by administering to a patient in need thereof an effective amount of:

a polypeptide comprising up to 50 amino acid residues, said polypeptide comprising a sequence of amino acids derived from the C-terminal E peptide of a Mechano Growth Factor (MGF) isoform of Insulin-like Growth Factor I (IGF-I); or an extended polypeptide comprising said polypeptide and extended by non-wild-type amino acid sequence N-terminal and/or C-terminal to said polypeptide; and said polypeptide or extended polypeptide possessing biological activity.

The invention also provides a method of treating a disorder of cardiac muscle by administering to a patient in need thereof an effective amount of:

a polypeptide comprising up to 50 amino acid residues, said polypeptide comprising a sequence of amino acids derived from the C-terminal E peptide of a Mechano Growth Factor (MGF) isoform of Insulin-like Growth Factor I (IGF-I); or an extended polypeptide comprising said polypeptide and extended by non-wild-type amino acid sequence N-terminal and/or C-terminal to said polypeptide;

and said polypeptide possessing biological activity.

The invention also provides use of

a polypeptide comprising up to 50 amino acid residues, said polypeptide comprising a sequence of amino acids derived from the C-terminal E peptide of a Mechano Growth Factor (MGF) isoform of Insulin-like Growth Factor I (IGF-I); or an extended polypeptide comprising said polypeptide and extended by non-wild-type amino acid sequence N-terminal and/or C-terminal to said polypeptide;

and said polypeptide possessing biological activity in the manufacture of a medicament for use in the treatment of a neurological disorder or a disorder of cardiac muscle.

### BRIEF DESCRIPTION OF THE DRAWINGS

FIG. 1: Sequence alignment, showing sequences encoded by part of the sequence of each of human, rat and rabbit MGF and human, rat and rabbit liver-type IGF-I (Amino acids 26 to 110 of SEQ ID NO: 2 and to 26 to 111 of SEQ ID NOS: 4 and 6: see below), and highlighting differences between MGF and liver-type IGF-I at C-terminus; created by 49 base pair insert in human MGF and 52 base pair insert in rat/rabbit MGF, leading to reading frame shift and divergence at C-terminus.

FIG. 2: Effect of Alanine substitution and C-terminal and N-terminal truncation on stability and biological activity—further sequence alignment, comparing modified sequences of Peptides 1-6 (SEQ ID NOS: 15-20) and Short peptides 1-4 (SEQ NOS: 21-24), and detailing impact of changes on stability as measured by incubation in human plasma and biological activity as measured by testing on muscle cell line (see Examples for details of test procedures).

In the Figure, the first two columns on the left hand side identify the peptides and give their sequences, identifying the changes made by way of substitution. The third column gives the results of the tests for stability (see Example 5 for details) and the final one on the right hand side gives the results of the tests for biological activity (again, see Example 5 for details).

FIG. 3: Increase in strength of a murine dystrophic muscle following injection of stabilised peptide after 3 weeks—

(A) percentage change in tetanic force in dystrophic muscle of mdx mice following injection of stabilised peptide (left hand column) and IGF (right hand column).

(B) percentage change in tetanic force in dystrophic muscle of mdx mice following injection of stabilised peptide (left hand column) and PBS vehicle control (right hand column).

FIG. 4: Cardioprotection following administration of stabilised peptide—comparison of ejection fractions achieved following administration to infarcted ovine heart of stabilised peptide (third column, referred to as “Ec domain”), full length MGF (fourth column), mature IGF-I (second column) and control preparation (first column).

FIG. 5: Pressure/volume loop data showing preservation of function following myocardial in fraction (MI)—for normal (top left) and infarcted (MI) murine (top right) ventricle, and showing effect of stabilised peptide delivered systemically to the MI heart (bottom right, referred to as “MGF peptide”) and the normal heart (bottom left). All panels show pressure (mmHg) on the Y-axis and Relative Volume Units on the X-axis.

FIG. 6: Neuroprotective effects in rat brain slice system—from left to right, percentage of dead cells after treatment with stabilised peptide (referred to as “MGF”), IGF-I, TBH, TBH+stabilised peptide (24 hours), TBH+IGF-I (24 hours), TBH+stabilised peptide (48 hours), TBH+IGF-I (48 hours).

FIG. 7: Western blots demonstrating the greater stability of the stabilised peptide that incorporates conversion of Arginine from L to D form and N-terminal PEGylation—the stability of the stabilised peptide compared to a corresponding one lacking the L to D form conversions and N-terminal PEGylation was investigated by incubation in fresh human plasma for a range of different time intervals. Western blotting was then used to assess the survival of each peptide over those time intervals: A=0 minutes; B=30 minutes; C=2 hours; D=24 hours. The results for the peptide with L-D conversion and N-terminal PEGylation are shown on the right; those for the peptide lacking the L to D form conversions and N-terminal PEGylation are on the left.

FIG. 8: Effect of 8 amino acid C-terminal peptides on proliferation of C2C12 muscle cells:

(A) DMGF and CMGF Peptides: C2C12 Cells were provided at 2000 cell/well, in a medium containing DMEM (1000 mg/L glucose), plus BSA(100 ug/ml), plus IGF-I (2 ng per ml) and incubated for 36 hours. Cell proliferation was then assessed using an Alamar Blue assay. The left hand group of readings shows the results for experiments with concentrations of the DMGF peptide (See Example 1.3.1 for details) of 2, 5, 50 and 100 ng/ml. The middle group of readings shows the results for experiments with concentrations of the CMGF peptide (See Example 1.3.1 for details) of 2, 5, 50 and 100 ng/ml. The left hand group of readings shows the results for experiments with concentrations of IGF-I alone (See Example 1.5 for details) of 2, 5, 50 and 100 ng/ml. Y-axis values are fluorescence (wavelength of excitation 535 nm, measurement at 590 nm; mean plus standard error) in an Alamar Blue assay.

(B) Peptides A2, A4, A6 and A8: C2C12 muscle cells at a 500 cells/well. Cultivation was carried out for 24 hours in 10% FBS, followed by starvation for 24 hours in 0.1% BSA, stimulation for 24 hours and then treatment with BrdU for 5 hours. Concentrations of 0.1, 1, 10 and 100 ng/ml of peptides A2, A4, A6 and A8 were tested, along with 0.1, 1, 10 and 100 ng/ml IGF-I (See the right-hand set of results). BrdU incorporation was measured to assess the level of cell proliferation achieved. Controls containing no cells, medium only, 5% FBS and no BrdU were also provided. Values on the Y-axis are for fluorescence (absorbence at 370 nm; mean plus standard error across 4 wells). The first column on the left relates to a control in which no cells were present. The next four relate to peptide A2 at concentrations of 0.1, 1, 10 and 100 ng/ml. The next four relate to peptide A4 at concentrations of 0.1, 1, 10 and 100 ng/ml. The central three relate to controls containing medium only (med), 5% FBS) and no BrdU. The next four relate to peptide A6 at concentrations of 0.1, 1, 10 and 100 ng/ml. The next four relate to peptide A8 at concentrations of 0.1, 1, 10 and 100 ng/ml. The right-hand group of results relate to IGF-I (See Example 1.5) at concentrations of 0.1, 1, 10 and 100 ng/ml.

FIG. 9: Effect on proliferation on HSMM cells

(A) Peptide A5: HSMM cells at 500 cells/well. Cultivation was carried out for 24 hours in 10% FCS, followed by two washes in serum free medium, stimulation for 48 hours and then treatment with BrdU for 5 hours. Concentrations of 0.1, 1, 10, 100 and 500 ng/ml of peptide A5 were tested, along with 0.1, 1, 10 and 100 ng/ml IGF-I. BrdU incorporation was measured to assess the level of cell proliferation achieved. Controls containing medium only, no cells (BLK), background staining (BG) and 10% FBS were also provided. Values on the Y-axis are for fluorescence (absorbence at 370 nm; mean plus standard error across 4 wells). The first five columns relate to peptide A5 at concentrations of 0.1, 1, 10, 100 and 500 ng/ml. The next column relates to the control containing medium only. The next three relate to IGF-I (See Example 1.5) alone at concentrations of 100, 10 and 0.1 ng/ml. The next three relate to controls containing 10% FBS, background staining and no cells respectively. * means P<0.05 compared to medium only control.

(B) Peptide AS: HSMM cells at 500 cells/well. Cultivation was carried out for 24 hours in 10% FCS, followed by two washes in serum free medium, stimulation for 48 hours and then treatment with BrdU for 5 hours. Concentrations of 0.1, 1, 10, 100 and 500 ng/ml of peptide AS in combination with 2 ng/ml IGF-I were tested, along with 0.1, 1, 10 and 100 ng/ml IGF-I. BrdU incorporation was measured to assess the level of cell proliferation achieved. Controls containing medium supplemented with 2 ng/ml IGF-I, no cells (BLK), and 10% FBS were also provided. Values on the Y-axis are for fluorescence (absorbence at 370 nm; mean plus standard error across 4 wells). The first five columns relate to peptide AS at concentrations of 0.1, 1, 10, 100 and 500 ng/ml. The next three relate to IGF-I (See Example 1.5) alone at concentrations of 100, 10 and 0.1 ng/ml. The next three relate to controls containing 10% FBS, medium supplemented with 2 ng/ml IGF-I and no cells respectively. * means P<0.01 and ** means P<0.001 compared to medium control containing 2 ng/ml IGF-I.

FIG. 10: Effect on proliferation on HSMM cells

(A) Peptide AS: HSMM cells at 500 cells/well. Cultivation was carried out for 24 hours in 10% FCS, followed by two washes in serum free medium, stimulation for 48 hours and then treatment with BrdU for 5 hours. Concentrations of 0.1, 1, 10, 100 and 500 ng/ml of peptide AS were tested, along with 0.1, 1, 10 and 100 ng/ml IGF-I. BrdU incorporation was measured to assess the level of cell proliferation achieved. Controls containing medium only, no cells (BLK), background staining (BG) and 10% FBS were also provided. Values on the Y-axis are for fluorescence (absorbence at 370 nm; mean plus standard error across 4 wells). The first five columns relate to peptide A5 at concentrations of 0.1, 1, 10, 100 and 500 ng/ml. The next column relates to the control containing medium only. The next three relate to IGF-I (See Example 1.5) alone at concentrations of 100, 10 and 0.1 ng/ml. The next three relate to controls containing 10% FBS, background staining and no cells respectively. * means P<0.05 compared to medium only control.

(B) Peptide A5: HSMM cells at 500 cells/well. Cultivation was carried out for 24 hours in 10% FCS, followed by two washes in serum free medium, stimulation for 48 hours and then treatment with BrdU for 5 hours. Concentrations of 0.1, 1, 10, 100 and 500 ng/ml of peptide AS in combination with 2 ng/ml IGF-I were tested, along with 0.1, 1, 10 and 100 ng/ml IGF-I. BrdU incorporation was measured to assess the level of cell proliferation achieved. Controls containing medium supplemented with 2 ng/ml IGF-I, no cells (BLK), background staining (BG) and 10% FBS were also provided. Values on the Y-axis are for fluorescence (absorbence at 370 nm; mean plus standard error across 4 wells). The first five columns relate to peptide AS at concentrations of 0.1, 1, 10, 100 and 500 ng/ml. The next column relates to the control containing medium supplemented with 2 ng/ml IGF-I only. The next three relate to IGF-I (See Example 1.5) alone at concentrations of 100, 10 and 0.1 ng/ml. The next three relate to controls containing 10% FBS, background staining and no cells respectively. * means P<0.1 compared to medium control containing 2 ng/ml IGF-I.

FIG. 11: Effect on proliferation on HSMM cells (A) Peptide AS: HSMM cells at 1000 cells/well. Cultivation was carried out for 24 hours in 10% FCS, followed by two washes in serum free medium, stimulation for 48 hours and then treatment with BrdU for 5 hours. Concentrations of 0.1, 1, 10, 100 and 500 ng/ml of peptide AS were tested, along with 0.1, 1, 10 and 100 ng/ml IGF-I. BrdU incorporation was measured to assess the level of cell proliferation achieved. Controls containing medium only, no cells (BLk), background staining (BG) and 10% FCS were also provided. Values on the Y-axis are for fluorescence (absorbence at 370 nm; mean plus standard error across 4 wells). The first five columns relate to peptide A5 at concentrations of 0.1, 1, 10, 100 and 500 ng/ml. The next column relates to the control containing medium only. The next three relate to IGF-I (See Example 1.5) alone at concentrations of 100, 10 and 0.1 ng/ml. The next three relate to controls containing 10% FCS, background staining and no cells respectively.

(B) Peptide A5: HSMM cells at 1000 cells/well. Cultivation was carried out for 24 hours in 10% FCS, followed by two washes in serum free medium, stimulation for 48 hours and then treatment with BrdU for 5 hours. Concentrations of 0.1, 1, 10, 100 and 500 ng/ml of peptide A5 in combination with 2 ng/ml IGF-I were tested, along with 0.1, 1, 10 and 100 ng/ml IGF-I. BrdU incorporation was measured to assess the level of cell proliferation achieved. Controls containing medium supplemented with 2 ng/ml IGF-I, no cells (BLK), background staining (BG) and 10% FBS were also provided. Values on the Y-axis are for fluorescence (absorbence at 370 nm; mean plus standard error across 4 wells). The first five columns relate to peptide A5 at concentrations of 0.1, 1, 10, 100 and 500 ng/ml. The next column relates to the control containing medium supplemented with 2 ng/ml IGF-I only. The next three relate to IGF-I (See Example 1.5) alone at concentrations of 100, 10 and 0.1 ng/ml. The next three relate to controls containing 10% FBS, background staining and no cells respectively. * means P<0.1 compared to medium control containing 2 ng/ml IGE-I.

Sequence Information

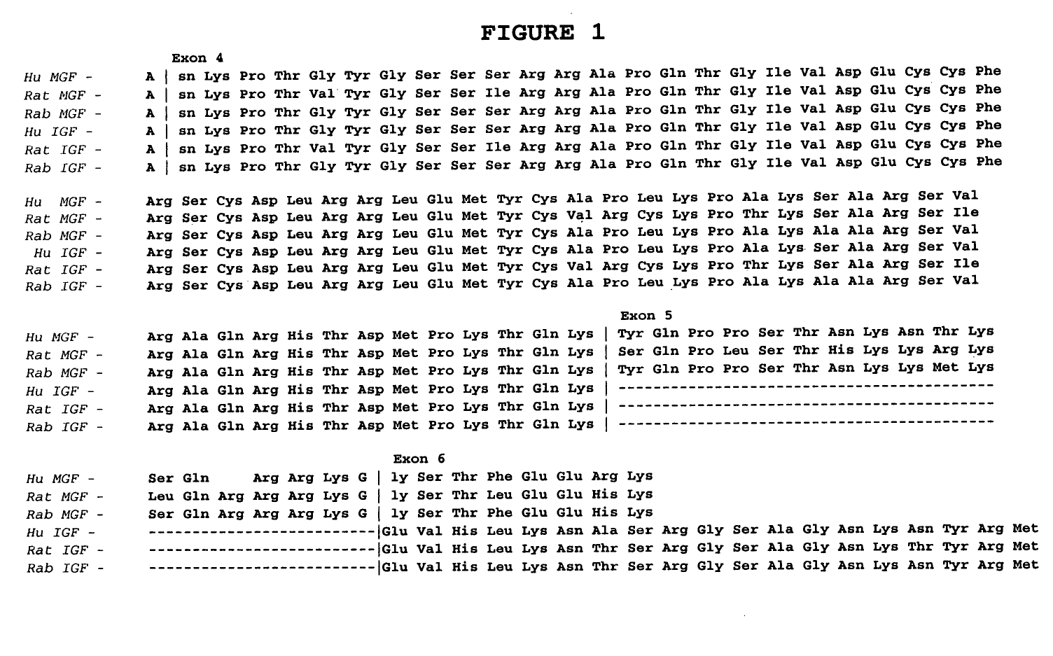

The DNA and amino acid sequences of human, rat and rabbit MGF DNA and are given in the sequence listing as SEQ ID NOS: 1/2, 3/4 and 5/6 respectively. These are termed full-length MGF sequences in that they represent mature MGF encoded by exons 3/4/5/6 of the IGF-I gene, including the 49/52 base pair insert that changes the reading frame and creates the characteristic MGF C-terminus. Exons 1 and 2 are alternative leader sequences. For comparison, the corresponding DNA and amino acid sequences from human, rat and rabbit liver-type IGF-I are given as SEQ ID NOS: 7/8, 9/10 and 11/12 respectively. A comparison of the six amino acid sequences, from the beginning of the sequence encoded by exon 4 onwards, is made in FIG. 1.

The sequence of the native rat Eb peptide (25 amino acids; amino acids 87-111 of SEQ ID NO: 4) from the C-terminus of rat MGF is given as SEQ ID NO: 13.

The sequence of the native rabbit Eb peptide (25 amino acids; amino acids 87-111 of SEQ ID NO: 6) from the C-terminus of rabbit MGF is given as SEQ ID NO: 14.

The sequence of the native human Ec peptide (24 amino acids; amino acids 87-110 of SEQ ID NO: 2) from the C-terminus of human MGF is given as SEQ ID NO: 27.

Modified sequences derived from the peptide of SEQ ID NO: 27 are given as SEQ ID NOS: 28 to 32.

In SEQ ID NO: 28, Serine is replaced with Alanine at position 5.

In SEQ ID NO: 29, Serine is replaced with Alanine at position 12.

In SEQ ID NO: 30, Serine is replaced with Alanine at position 18.

In SEQ ID NO: 31, Arginine is replaced with Alanine at position 14.

In SEQ ID NO: 32, Arginine is replaced with Alanine at position 14 and Arginine is also replaced with Alanine at position 15.

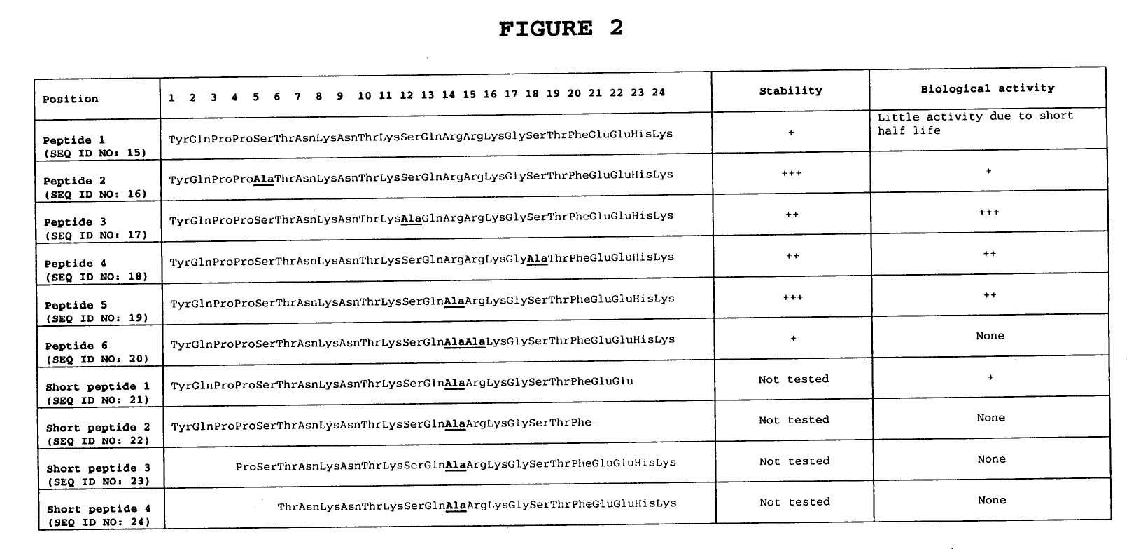

Native human Ec peptide has Arginine in its penultimate position. A variant of the native peptide with Histidine in the penultimate position has been synthesised and is shown in SEQ ID NO: 15. This peptide is also described as Peptide 1 in FIG. 2. SEQ ID NO: 26 represents the sequence of full-length human MGF incorporating Histidine in the penultimate position instead of Arginine. SEQ ID NO: 25 is a DNA coding sequence for SEQ ID NO: 26, in which the Histidine in the penultimate position is encoded by CAC and the remaining sequence is the same as in SEQ ID NO: 1.

Modified sequences derived from the peptide of SEQ ID NO: 15 are given as SEQ ID NOS: 16 to 24. These are compared to peptide of SEQ ID NO: 15 and one another in FIG. 2.

In Peptide 2 (SEQ ID NO: 16), Serine is replaced with Alanine at position 5.

In Peptide 3 (SEQ ID NO: 17), Serine is replaced with Alanine at position 12.

In Peptide 4 (SEQ ID NO: 18), Serine is replaced with Alanine at position 18.

In Peptide 5 (SEQ ID NO: 19), Arginine is replaced with Alanine at position 14.

In Peptide 6 (SEQ ID NO: 20), Arginine is replaced with Alanine at position 14 and Arginine is also replaced with Alanine at position 15.

In Short peptide 1 (SEQ ID NO: 21), Arginine is replaced with Alanine at position 14 and the two C-terminal amino acids are removed.

In Short peptide 2 (SEQ ID NO: 22), Arginine is replaced with Alanine at position 14 and the four C-terminal amino acids are removed.

In Short peptide 3 (SEQ ID NO: 23), Arginine is replaced with Alanine at position 14 and the three N-terminal amino acids are removed.

In Short peptide 4 (SEQ ID NO: 24), Arginine is replaced with Alanine at position 14 and the five N-terminal amino acids are removed.

Four 8 amino acid peptide sequences are also included in the Sequence Listing.

SEQ ID NO: 33 is the 8 C-terminal amino acids of the variant sequence of SEQ ID NO:15, containing Histidine in the penultimate position.

SEQ ID NO: 34 is the 8 C-terminal amino acids of the native human MGF C-terminus of SEQ ID NO:27, containing Arginine in the penultimate position.

SEQ ID NO: 35 is the sequence of SEQ ID NO: 33 with Serine in position 2 substituted with Alanine. This therefore corresponds to the 8 C-terminal amino acids of SEQ ID NO: 18 (Peptide 4).

SEQ ID NO: 36 is the sequence of SEQ ID NO: 34 with Serine in position 2 substituted with Alanine. This therefore corresponds to the 8 C-terminal amino acids of SEQ ID NO: 30.

For ease of reference, these sequences are also described in the following Table.

| SEQ ID | Description |
|---|---|
| NO: | (“aa” denotes “amino acid”) |
| 1 | Full length human IGF-1-Ec (= MGF) (Nucleotide and amino acid) |
| 2 | Full length human IGF-1-Ec (= MGF) (Amino acid only) |
| 3 | Full length rat IGF-1-Eb (≡ rat MGF) (Nucleotide and amino acid) |
| 4 | Full length rat IGF-1-Eb (≡ rat MGF) (Amino acid only) |
| 5 | Full length rabbit IGF-1-Eb (≡ rabbit MGF) (Nucleotide and amino acid) |
| 6 | Full length rabbit IGF-1-Eb (≡ rabbitMGF) (Amino acid only) |
| 7 | Full length human liver-type IGF-1 (Nucleotide and amino acid) |
| 8 | Full length human liver-type IGF-1 (Amino acid only) |
| 9 | Full length rat liver-type IGF-1 (Nucleotide and amino acid) |
| 10 | Full length rat liver-type IGF-1 (Amino acid only) |
| 11 | Full length rabbit liver-type IGF-1 (Nucleotide and amino acid) |
| 12 | Full length rabbit liver-type IGF-1 (Amino acid only) |
| 13 | Synthetic peptide corresponding to aa 87-111 of SEQ ID NO: 4 |
| 14 | Synthetic peptide corresponding to aa 87-111 of SEQ ID NO: 6 |
| 15 | Synthetic peptide corresponding to aa 87-110 of SEQ ID NO: 2 with |
| | Arg109→His (= Arg23→His using SEQ ID NO: 15 numbering) |
| 16 | Peptide of SEQ ID NO: 15 with Ser5→Ala |
| 17 | Peptide of SEQ ID NO: 15 with Ser12→Ala |
| 18 | Peptide of SEQ ID NO: 15 with Ser18→Ala |
| 19 | Peptide of SEQ ID NO: 15 with Arg14→Ala |
| 20 | Peptide of SEQ ID NO: 15 with Arg14→Ala, Arg15→Ala |
| 21 | Synthetic peptide corresponding to aa 1-22 of SEQ ID NO: 15 with Arg14→Ala |
| 22 | Synthetic peptide corresponding to aa 1-20 of SEQ ID NO: 15 with Arg14→Ala |
| 23 | Synthetic peptide corresponding to aa 4-24 of SEQ ID NO: 15 with |
| | Arg14→Ala and Arg23→His |
| 24 | Synthetic peptide corresponding to aa 6-24 of SEQ ID NO: 2 with |
| | Arg14→Ala and Arg23→His |
| 25 | Sequence of SEQ ID NO: 1 with Arg109→His (Nucleotide and amino acid) |
| 26 | Sequence of SEQ ID NO: 2 with Arg109→His (Amino acid only) |
| 27 | Synthetic peptide corresponding to aa 87-110 of SEQ ID NO: 2 |
| 28 | Peptide of SEQ ID NO: 27 with Ser5→Ala |
| 29 | Peptide of SEQ ID NO: 27 with Ser12→Ala |
| 30 | Peptide of SEQ ID NO: 27 with Ser18→Ala |
| 31 | Peptide of SEQ ID NO: 27 with Arg14→Ala |
| 32 | Peptide of SEQ ID NO: 27 with Arg14→Ala, Arg15→Ala |
| 33 | Peptide corresponding to the 8 C-terminal amino acids of SEQ ID NO: 15 |
| 34 | Peptide corresponding to the 8 C-terminal amino acids of SEQ ID NO: 27 |
| 35 | Peptide corresponding to the 8 C-terminal amino acids of SEQ ID NO: 18 |
| 36 | Peptide corresponding to the 8 C-terminal amino acids of SEQ ID NO: 30 |

### DETAILED DESCRIPTION OF THE INVENTION

Polypeptides and Extended Polypeptides of the Invention

Polypeptides of the Invention

Polypeptides of the invention are up to 50 amino acid residues in length. For example, they may be up to 10 amino acids in length, up to 30 amino acids in length, e.g. 11, 12, 13, 14, 15, 16, 17, 18, 19, 20, 21, 22, 23, 24, 25, 26, 27, 28, 29 or 30 amino acids in length, or up to 35, 40, 45 or 50 amino acids in length. Preferably, they are from 15 to 30 amino acids in length, more preferably 20 to 28, most preferably 22, 23, 24 or 25 amino acids in length. Also preferred are polypeptides of 5 to 10 amino acids in length, i.e. 5, 6, 7, 8, 9 or 10 amino acids in length, especially those of 8 amino acids in length.

A polypeptide of the invention comprises a sequence of amino acids derived from the C-terminal E peptide of an MGF isoform of IGF-I. An MGF isoform is, as discussed above, one in which alternative splicing introduces into the mRNA an insert which lengthens and changes the reading frame of the C-terminal E peptide found at the C-terminus of IGF-I to create an Ec or Eb peptide. An MGF isoform will typically have at least 80%, preferably 85% or 90% sequence identity to one of the MGFs of SEQ ID NOS: 2, 4, or 6. In human MGF (SEQ ID NOS: 1 and 2), the insert is 49 base pairs and the C-terminal E peptide is known as an Ec peptide (SEQ ID NO: 27), which is 24 amino acids in length. In rat and rabbit MGF (SEQ ID NOS: 3-6), the insert is 49 base pairs and the C-terminal E peptides are known as Eb peptides, which are 25 amino acids in length (SEQ ID NOS: 13 and 14). The sequence of the invention may be derived from any of these MGF C-terminal E peptides or from any other C-terminal E peptide from the MGF of any other species.

The sequence comprised in the polypeptide of the invention and derived from the C-terminal E peptide of an MGF isoform may be derived from said C-terminal E peptide in any way, as long as the requirements for biological activity and stability (see below) are met. In particular, the sequence may be derived from the MGF C-terminal E peptide in the sense that it has exactly the sequence of the C-terminal E peptide (e.g. SEQ ID NO: 13, 14, 27 or 34) and is merely not present within a full-length MGF molecule. It may also be derived from the MGF C-terminal E peptide in the sense that its sequence is altered (see “Modifications” below), again as long as the requirements for biological activity and stability (see below) are met.

Up to the maximum length of 50 amino acids, the polypeptide may also comprise native MGF sequence N-terminal to the sequence derived from the C-terminal E peptide. Alternatively, any additional sequence may be non-MGF-derived, i.e. it may be any sequence, again as long as the requirements for biological activity and stability (see below) are met.

The sequence derived from the C-terminal MGF E peptide may include at least 10, at least 15 or at least 20 amino acids, e.g. 15, 16, 17, 18, 19, 20, 21, 22, 23 or 24 amino acids in the case of the human C-terminal MGF Ec peptide or 15, 16, 17, 18, 19, 20, 21, 22, 23, 24 or 25 amino acids in the case of the rat or rabbit C-terminal MGF Eb peptide. Alternatively, it may include up to 10 amino acids, preferably 5 to 10 amino acids, ie 5, 6, 7, 8, 9 or 10 amino acids, especially 8 amino acids.

Polypeptides or extended polypeptides of the invention can be assembled together to form larger structures containing two or more polypeptide of the invention, e.g. multiple copies of the same polypeptide or extended polypeptides of the invention or a mixture of different ones. Depending on the nature of the polypeptides and in particular whether they contain any L-D conversions (see below), these structures may be made as fusion proteins, normally by recombinant expression by standard techniques from coding DNA, or assembled synthetically, or expressed as fusion proteins and then subjected to appropriate chemical modifications.

Extended Polypeptides of the Invention

An extended polypeptide of the invention comprises a polypeptide of the invention, extended by non-wild-type sequence. By this is meant that any extension sequence is non-MGF sequence in that, if the N-terminus or C-terminus of the polypeptide of the invention represents native MGF sequence, then that sequence may not simply be joined to any sequence that it adjoins in native MGF. Apart from that, an extension may have any sequence. Thus, the polypeptides of the invention may be extended at either or both of the C- and N-termini by an amino acid sequence of any length. For example, an extension may comprise up to 5, up to 10, up to 20, up to 50, or up to 100 or 200 or more amino acids. Typically, any such extension will be short, e.g. 1, 2, 3, 4, 5, 6, 7, 8, 9 or 10 amino acids in length. An extension may contain, or even consist entirely of D-form amino acids (see below), e.g. to reduce exopeptidase attack. For example, a polypeptide may be extended by 1 to 5 D-form amino acids at one or both ends. For example, in some embodiments an additional Cysteine residue may be incorporated at the C-terminus.

Modifications

A polypeptide or extended polypeptide of the invention may be modified in any manner that increases its stability compared to the unmodified E peptide that they comprise a sequence derived from. Stability may be increased in various ways. For example, it is envisaged that modifications (e.g. PEGylation or other chemical modifications or L-D form amino acid conversions) to the C- and/or N-termini of the protein will protect it against exopeptidase attack, as will cyclisation, and that internal modifications (e.g. substitution, deletion, insertion and internal L-D form conversion will protect it against cleavage by endopeptidases by disrupting their cleavage sites.

For example, it may be PEGylated, preferably at the N-terminus to the extent that the location of the PEGylation can be controlled, though PEGylation at other sites, such as the C-terminus and between the C- and N-termini is also contemplated. PEGylation involves the covalent attachment of PEG to the polypeptide. Any suitable type of PEG, e.g. any suitable molecular weight, may be used as long as the resultant PEGylated polypeptide satisfies the requirements for biological activity and stability (see below).

Whether to achieve stabilisation or otherwise, polypeptide of the invention may also incorporate other chemical modifications as well as, or instead of, PEGylation. Such modifications include glycosylation, sulphation, amidation and acetylation. In particular, polypeptides may be acetylated at the N-terminus are preferred or amidated at the C-terminus or both. Alternatively or additionally, one or more hexanoic or amino-hexanoic acid moieties may be added, preferably one hexanoic or amino-hexanoic acid moiety, normally at the N-terminus.

In addition or alternatively, the polypeptide or extended polypeptide may include one or more D-form amino acids. In nature, amino acids are in the L-form. Inserting D-form amino acids can improve stability. Typically, a few, e.g. 1, 2, 3, 4 or 5, D-form amino acids may be used. However, more can also be used, e.g. 5 to 10, 10 to 15, 15 to 20 or 20 or more as long as the resultant PEGylated polypeptide satisfies the requirements for biological activity and stability (see below). If those requirements are satisfied, the entire polypeptide may even be synthesised using D-form amino acids.

D-form amino acids may be used at any position in the polypeptide. In the human MGF C-terminal E peptide of SEQ ID NO: 27, it is preferred to replace one or both of the Arginines at positions 14 and 15 with D-form amino acids. Corresponding changes are also preferred in the rat and rabbit sequences of SEQ ID NOS: 13 and 14 (positions 14, 15 and 16, as the rat/rabbit sequences comprise three Arginines in succession whereas the human one has only two) and in the variant sequence of SEQ ID NO: 15.

Stereochemical and/or directional peptide isomers may also be used. For example, Retro (RE) peptides may be used, in which the sequence of the invention is assembled from L-amino acids but in reversed order. Alternatively, Retro-inverso (R1) peptides may be used, in which the sequence is reversed and synthesised from D-amino acids.

Additionally or alternatively, D-form amino acids may be included at one end or the other, or both, of the polypeptide. It is envisaged that this will help to protect against exopeptidase attack. This may be achieved by converting the terminal amino acids, e.g. the terminal 1, 2, 3, 4 or 5 amino acids at one or both ends, of the sequence derived from the MGF C-terminal E peptide to D-form. Alternatively or additionally, it may be achieved by adding 1, 2, 3, 4 or 5 further D-form amino acids at one or both ends of the polypeptide. Such further amino acids may or may not correspond to those that adjoin the sequence derived from the MGF C-terminal E peptide in native MGF. Such further amino acids may be any amino acids. One possible amino acid for addition in D-form in this way is Arginine. For example, a D-form Arginine residue may be added at the N-terminus, the C-terminus or both.

In one embodiment, the sequence of the native human MGF C-terminal E peptide of SEQ ID NO: 27 is retained but the Arginines at positions 14 and 15 of SEQ ID NO: 27 are converted to the D-form and N-terminal PEGylation is provided. C-terminal amidation may also be provided.

In another embodiment, the sequence of the human MGF C-terminal E peptide variant of SEQ ID NO: 15 is retained but Arginines 14 and 15 in SEQ ID NO: 15 are converted to the D-form and N-terminal PEGylation is provided.

In some further embodiments, the sequence of the 8 C-terminal amino acids from SEQ ID NO: 15 or 27, ie the sequence of SEQ ID NO: 33 or 34, is used and N-terminal PEGylation is provided or a hexanoic or amino-hexanoic acid moiety is added at the C-terminus. C-terminal amidation may also be provided.

Alternatively or additionally, polypeptides of the invention may also incorporate other modifications, for example truncation, insertion, internal deletion or substitution.

As to truncation, it has has also been found that shorter peptides, based on the C-terminal eight amino acids of SEQ ID NO: 15 are active. However, the results in Example 5 below suggest that the activity of longer peptides related to the MGF C-terminus can be quite sensitive to truncation, particularly of the N-terminus of the peptides. At the N-terminus of the peptide of SEQ ID NO: 15, truncation by 3 amino acids led to loss of activity in the muscle cell model used in Example 5. At the C-terminus of the peptide of SEQ ID NO: 15, truncation by four amino acids led to loss of activity in the muscle cell model, though truncation by two did not. In the case of the native human, rat and rabbit E peptide sequences, and in the variant one of SEQ ID NO: 15 and other peptides of the invention that have lengths comparable to those of the native peptides (eg 18 or more amino acids), it is therefore envisaged that it will be possible to truncate by 1, 2 or 3 amino acids at the C-terminus without loss of activity. It is also envisaged that it will be possible to truncate by 1 or 2 amino acids at the N-terminus without loss of activity.

As to insertion, short stretches of amino acids may be inserted into the sequence derived from that of human C-terminal MGF E peptide, as long as the resultant polypeptide satisfies the requirements for biological activity and stability (see below) and comprises less than 50 amino acids. Each insertion may comprise, for example 1, 2, 3, 4 or 5 amino acids. There may be one or more, e.g. 2, 3, 4 or 5 such insertions.

As to internal deletion, short stretches of amino acids may be deleted from the internal sequence derived from that of human C-terminal MGF E peptide, as long as the resultant polypeptide satisfies the requirements for biological activity and stability (see below). One or more such deletions, e.g. 1, 2, 3, 4 or 5 deletions, may be made, up to a total of, for example, 1, 2, 3, 4, 5, 6, 7, 8, 9 or 10 amino acids.

As to substitution, any amino acids in the polypeptide may in principle be substituted by any other amino acid, as, as long as the resultant polypeptide satisfies the requirements for biological activity and stability (see below). One or more such substitutions may be made, e.g. 1, 2, 3, 4, 5, 6, 7, 8, 9, 10, up to 15 or up to 20 substitutions in total. Preferably, in the sequence derived from the MGF C-terminal E peptide, no more than 10 substitutions will be made, e.g. 1, 2, 3, 4, 5, 6, 7, 8, 9 or 10 substitutions. Preferably, in the in the sequence derived from the MGF C-terminal E peptide, at least 50%, at least 60%, at least 70%, at least 80% or at least 90% of the amino acid residues will be the same as in the native MGF C-terminal E peptide from which the sequence is derived. In one preferred approach, residues at one or both ends of the polypeptide (terminal residues) are substituted. It is envisaged that this will protect against exopeptidase attack. Thus, for example, it may be preferred to substitute residues in the N-terminal and for C-terminal positions, or in the positions immediated adjacent to the terminal ones, or up to 3, 4 or 5 positions from one or both ends.

Substitutions may increase stability or biological activity. For example, the results discussed in Example 5 and FIG. 2 below indicate that substitution at one or more of positions 5, 12, 14 and 18 of the peptide of SEQ ID NO: 15 can increase stability.

The same results show that substitutions at positions 12, 14 and 18 can also increase biological activity. Substitutions in positions 5, 12, 14 and 18 of the peptides of SEQ ID NOS: 27 and 15, and in position 2 of SEQ ID NOS 33 and 34 (which corresponds to position 18 of SEQ ID NOS: a5 and 27), are therefore preferred. Corresponding substitutions into positions 5, 12, 15 and 19 of rat/rabbit MGF C-terminal E peptides of SEQ ID NOS: 13 and 14 are also preferred.

Whether in positions 5, 12, 14 or 18 of SEQ ID NOS: 27 or 15, position 2 of SEQ ID NOS: 33 and 34, positions 5, 12, 15 and 19 of SEQ ID NOS: 13 and 14, or elsewhere, substitution of the native amino acid with Alanine is one preferred option, as shown in Example 5 and FIG. 2. However, other amino acids may equally be used.

Alternatively or additionally, the polypeptide may include substitutions that do not have a significant effect on stability or biological activity. These will typically be conservative substitutions. Conservative substitutions may be made, for example according to the following table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Typically, amino acid sequence modifications such as L-D conversions, substitutions, insertions and deletions, in the polypeptides of the invention will be found in the sequence of amino acids that is derived from the MGF C-terminal E peptide. However, where the polypeptide contains additional MGF sequence, they may alternatively or additionally be found in that additional sequence. For example, if a polypeptide of the invention contains further MGF sequence that is N-terminal to the sequence of the E peptide (e.g. SEQ ID NO: 13, 14 or 27) in native MGF, modifications may be found in that sequence.

Alternatively or additionally, stability can also be increased by cyclisation of the polypeptides or extended polypeptides of the invention. It is envisaged that this will protect against exopeptidase attack.

Preferred polypeptides of the invention include the following.
(i) A peptide which is 24 amino acids in length and has the sequence of SEQ ID NO: 15 but is stabilised by converting the two Arginines of SEQ ID NO 15 (positions 14 and 15) from L-form to D-form and by N-terminal PEGylation.
(ii) A peptide as in (i) above but lacking PEGylation, ie having the sequence of SEQ ID NO: 15 but stabilised by converting the two Arginines of SEQ ID NO 15 (positions 14 and 15) from L-form to D-form.
(iii) The peptides described in Example 5 and FIG. 2 as Peptides 2, 3, 4 and 5 (SEQ ID NOS: 16 to 19).
(iv) The peptide described in Example 5 and FIG. 2 as Short peptide 1 (SEQ ID NO: 21), which has the sequence of SEQ ID NO: 19 (in which Arginine at position 14 is replaced by Alanine) but is truncated by 2 amino acids at the C-terminus.
(v) A peptide corresponding to that of (i) above but based on the native human C-terminal peptide of SEQ ID NO: 27, which contains Arginine rather than Histidine in the penultimate position, ie a peptide having the sequence of SEQ ID NO: 27 but stabilised by converting the two Arginines at positions 14 and 15 of SEQ ID NO 27 from L-form to D-form and by N-terminal PEGylation.
(vi) A peptide as in (v) above but lacking PEGylation, ie having the sequence of of SEQ ID NO: 27 but stabilised by converting the two Arginines at positions 14 and 15 of SEQ ID NO 27 from L-form to D-form.
(vii) Peptides corresponding to those of (iii) above but based on the native human C-terminal peptide of SEQ ID NO: 27, which contains Arginine rather than Histidine in the penultimate position; shown herein as SEQ ID NOS: 28 to 31.
(viii) Peptides of any of SEQ ID NOS 33-36 with N-terminal PEGylation or the attachment of an N-terminal hexanoic or amino-hexanoic acid moiety.
(ix) Any of the peptides of (i), (ii), (iii), (iv), (v), (vi), (vii), or (vii) above with C-terminal amidation, notably the peptides of (ii) and (vi) above with C-terminal amidation, i.e. peptides having the sequences of SEQ ID NOS: 15 and 27, with conversion of L-Arginine to D-Arginine at positions 14 and 15 and C-terminal amidation, but lacking PEGylation.
(x) Any of the peptides of (i), (ii), (iii), (iv), (v), (vi), (vii), (viii) or (ix) above with an additional Cysteine residue at the C-terminus.
(xi) Any of the peptides of (i), (ii), (iii), (iv), (v), (vi), (vii), (viii) or (ix) above with an additional D-form Arginine residue at the N-terminus.

Modifications according to the invention may confer additional advantages as well as increased stability. For example, they may confer increased therapeutic activity or be advantageous from an immunological standpoint (eg via reduced immunogenicity).

This applies in particular to modifications that involve L-D conversion and/or stereochemical and/or directional isomerism (see above).

Biological Activity

Polypeptides and extended polypeptides of the invention have biological activity. This activity may be selected from the following.

The ability to increase muscle strength in dystrophic and/or non-dystrophic skeletal muscle in mice, humans or other mammals (cf. Example 2 below). Preferably, a polypeptide or extended peptide of the invention will be able to increase muscle strength (e.g. as measured by maximum attainable tetanic force) by at least 5%, at least 10%, at least 20%, at least 25%, at least 30%, at least 50%, at least 75% or at least 100% in dystrophic and/or non-dystrophic muscle.

Cardioprotective ability in sheep, mice, humans or other mammals (cf. Example 3 below). Preferably, a polypeptide or extended polypeptide of the invention will have the ability to prevent or limit myocardial damage in an infarcted or mechanically overloaded heart. This can be measured by pressure/volume loops or by reference to the ability to increase ejection fraction compared to an infracted heart to which no polypeptide or extended polypeptide of the invention is administered. Preferably a polypeptide or extended polypeptide of the invention will have the ability to increase ejection fraction by at least 1%, at least 2%, at least 3%, at least 4%, at least 5%, at least 6%, at least 7%, at least 8%, at least 9% or by at least 10% or more.

Neuroprotective ability in vitro or in vivo in mice, gerbils, humans or other mammals (cf. Example 4 below). Preferably, a polypeptide or extended polypeptide of the invention will have the ability to reduce cell death in rat organotypic hippocampal cultures and/or other similar in vitro models. Preferably, following exposure to TBH or other another agent that induces oxidative stress or causes damage in other ways, a polypeptide or extended polypeptide of the invention will have the ability to reduce cell death in such models by at least 20%, at least 25%, at least 30%, at least 50%, at least 60%, at least 70%, at least 75%, at least 80%, at least 85%, or at least 90% or more. Alternatively or additionally, polypeptides or extended polypeptides of the invention may have neuroprotective ability

Furthermore, the polypeptides or extended polypeptides of the invention may have one or more biological properties characteristic of full-length MGF (e.g. of SEQ ID NOS: 2, 4 or 6). For example, polypeptides or extended polypeptides of the invention may have the functional properties of MGF identified in WO97/33997. In particular, they may have the ability to induce growth of skeletal muscle tissue. Similarly, as discussed herein, they may have the ability to upregulate protein synthesis needed for skeletal muscle repair and/or to activate satellite (stem) cells in skeletal muscle.

In this regard, one method of assessing biological activity is the Alamar Blue method as discussed in Example 5.2.2. This involves contacting a polypeptide with mononucleated myoblast cells and assessing the extent to which it causes them to proliferate. This can be scored in any suitable way, e.g. on a scale of 0 to 3 as discussed in the Examples. Activity may also be measured via cyclins, such as cyclin ID, which are early markers of cell division. Activity may also be measured via the use of bromodeoxy uridine (BrdU). BrdU will substitute itself for thymidine during DNA replication and hence can be used to identify cells whose DNA is undergoing replication and to measure how much replication and cell division is taking place.

Alternatively or additionally, polypeptides or extended polypeptides of the invention may have the neurological properties previously identified in WO01/136483. Thus, they may have the capacity to effect motoneurone rescue. In particular, they may be able to reduce motoneurone loss following nerve avulsion by up to 20, 30, 40, 50, 60, 70, 80, 90, 95, 99 or 100% in a treated subject compared to an equivalent situation in a non-treated subject. Reduction of motoneurone loss by 70% or more, or 80% more (i.e. to 30% or less or 20% or less) is preferred. The degree of rescue may be calculated using any suitable technique, e.g. a known technique such as Stereology. As a specific test, the techniques used in WO01/136483, which rely on measuring motoneurone rescue in response to facial nerve avulsion in rats, may be used.

Alternatively or additionally, polypeptides or extended polypeptides of the invention may have the properties identified in WO03/060882, which is to say the ability to prevent or limit myocardial damage following ischemia or mechanical overload by preventing cell death, or apoptosis, of the muscle cells of the myocardium. Preferably, a polypeptide or extended polypeptide of the invention will have the ability to completely prevent apoptosis in the area of cardiac muscle to which it is applied. However, apoptosis may also be only partially prevented, i.e. limited. Damage is limited if any reduction of damage is achieved compared to that which would have taken place without a treatment of the invention, e.g. if damage is reduced by 1% or more, 5% or more, 10% or more, 20% or more, 30% or more, 50% or more, 70% or more, 80% or more, 90% or more, 95% or more, 98% or more, or 99% or more, as measured by the number or proportion of cells which die, or by the size of the area of muscle that loses function, or by the overall ability of the heart to pump blood.

In particular, reduction of damage can be estimated in vivo by determining cardiac output, ejection fraction etc using minimally invasive methods. Markers such as creatine kinase and troponin T in the serum can also be assayed. These are the parameters used in clinical situations to determine the extent damage to the cardiac muscle following injury.

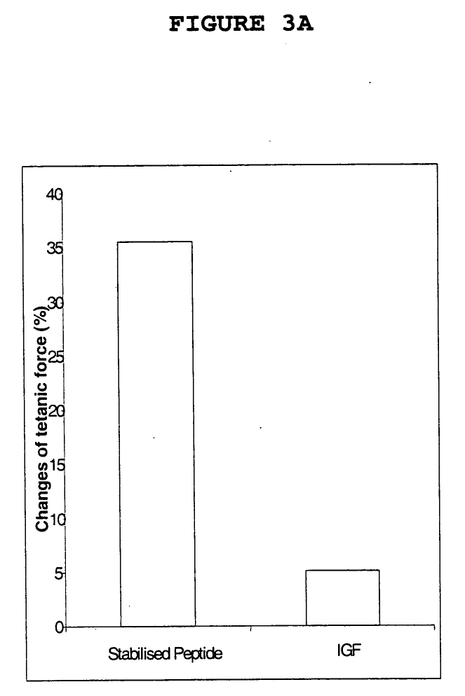

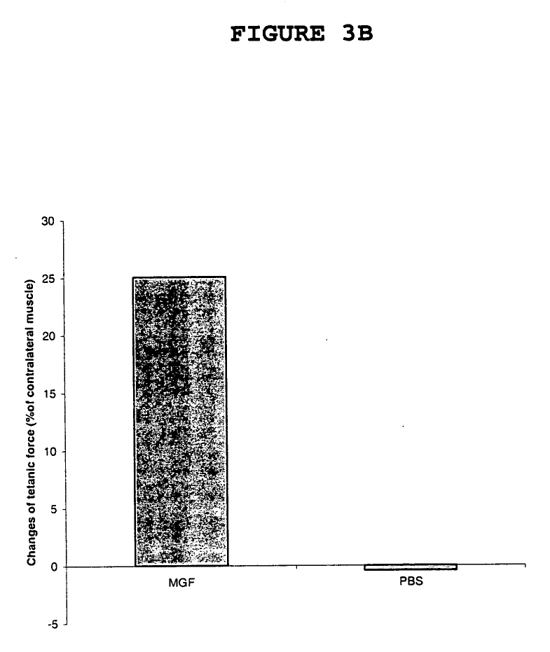

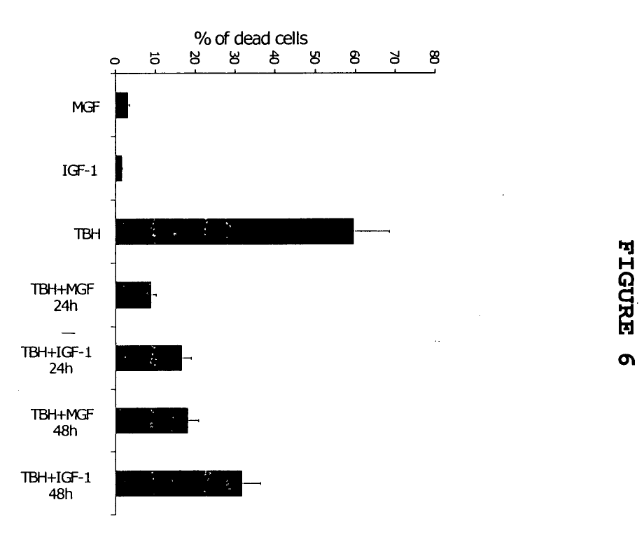

The ability to prevent apoptosis may be measured by any suitable technique. For example, with reference to Example 4 and FIGS. 3 and 6, it may be measured by the ability to prevent apoptosis in a cardiac muscle cell or cardiac-like cell line, as indicated by DNA fragmentation. The ability to prevent apoptosis, as indicated by DNA fragmentation, may be tested by treating the cells with sorbitol or another agent that places the cells under osmotic stress for up to, e.g. 1, 2, 4, 6, 12, 24 or 48 hours, preferably 12 to 24 hours, more preferably 24 hours, and investigating whether the pattern of fragmentation associated with apoptosis can be observed. An MGF polypeptide of the invention expressed in this way will typically reduce, preferably eliminate, DNA fragmentation under these conditions, as compared to an untreated cell) after 6, 12 or 24 hours' sorbitol treatment.

The absence of expression, or low expression, of genes that act as markers for apoptosis can also act as an indication of prevention of apoptosis. One suitable marker is the Bax gene. Similarly, increased expression of anti-apoptotic markers in MGF-transfected cells under apoptotic conditions can be taken as a sign that the polypeptide of the invention is preventing apoptosis. One suitable anti-apoptotic marker gene is Bcl2. The ability to prevent apoptosis may also be measured by reference to an MGF polypeptide's ability to prevent a reduction in cell number in myocyte cells in vitro.

Another preferred property of polypeptides and extended polypeptides of the invention is the ability to induce a hypertrophic phenotype in cardiac muscle cells. In particular, this may be tested by assessing the ability to induce a hypertrophic phenotype in primary cardiac myocyte cultures in vitro. A preferred method for determining this is to test for an increase in expression of ANF (Atrial Natriuretic Factor) and/or bMHC (Beta Myosin Heavy Chain). ANF is an embryonic marker gene that is upregulated in hypertrophic conditions. bMHC is an important contractile protein in muscle.

Stability of Polypeptides and Extended Polypeptides of the Invention

Polypeptides and extended polypeptides of the invention have increased stability compared to the native C-terminal MGF E peptides that they contain sequences derived from. Such comparisons are made between the polypeptide or extended polypeptide of the invention and the native C-terminal MGF E peptide in its isolated, unmodified form (e.g. an unmodified form of SEQ ID NO: 13, 14, 27, or 34, separated from the remainder of the MGF molecule and in isolated form as a 24-mer (SEQ ID NO: 27), 25-mer (SEQ ID NOS 13/14) or 8-mer (SEQ ID NO 34)). Comparisons may also be made with the Histidine-containing sequences of SEQ ID NO: 15 and 33. Stability may be increased by any degree via the modifications discussed herein.

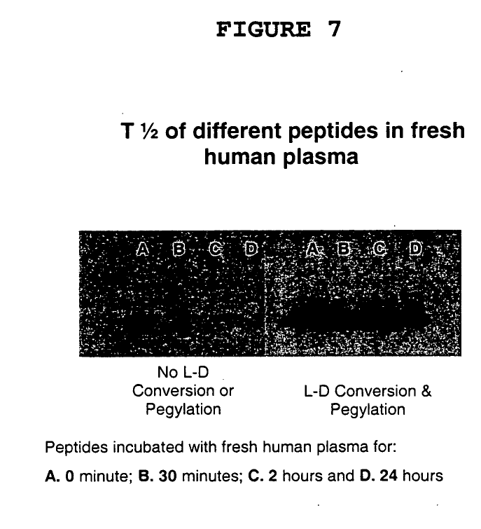

Stability may be assessed in terms of half-life in human plasma or by any other suitable technique. In particular, stability can be measured by assessing peptides' susceptibility to proteolytic cleavage in fresh human plasma according to the technique of Example 5.1 below, in which the plasma was stored until used at −70° C., 10 μg of peptide was added to 2 ml of plasma, plus 7 ml of PBS and the mixture was incubated at 37° C. for different time intervals. Western blotting was then used to detect each peptide over those time intervals. (In FIG. 7: A=0 minutes; B=30 minutes; C=2 hours; D=24 hours. The results for the peptide with L-D conversion and N-terminal PEGylation are shown on the right; those for the peptide lacking the L to D form conversions and N-terminal PEGylation are on the left.) Relatively little of the peptide lacking L-D conversion and PEGylation could be detected after 30 minutes, very little after 2 hours and none or almost none after 24 hours. In contrast, the peptide with L-D conversion and PEGylation could be detected in much greater abundance and 2 hours and 24 hours.

Other measures of stability can be based on determining the loss of biological activity over time. This can be done by any suitable method, e.g. via an in vitro assay for any of the measures of biological activity discussed herein.

Quantitatively, in relative terms, preferred polypeptides or extended polypeptides of the invention may have half-lives that are increased by at least 10%, at least 20%, at least 30%, at least 50%, at least 60%, at least 80%, at least 100%, at least 200% or at least 500% or more compared to the corresponding unmodified MGF C-terminal E peptide.

Quantitatively, in absolute terms, preferred polypeptides or extended polypeptides of the invention may have half-lives of at least 1 hour, at least 2 hours, at least 4 hours, at least 8 hours, at last 12 hours, at least 24 hours or at least 48 hours or more.

Alternatively, qualitative or semi-quantitative measurements of stability may be used, as in Example 5 and FIG. 2, for example by scoring the stability of polypeptides or extended polypeptides on a scale from 0 to 3. On that scale, the polypeptide of SEQ ID NO: 15 scored 1. Certain other modified polypeptides of the invention scored 2 or 3. A polypeptide or extended polypeptide of the invention will generally score more highly on such a scale than the corresponding native MGF C-terminal E peptide.

Further Peptides of the Invention

Whilst many of the peptides of the invention will be stabilised, as discussed above, it may under certain circumstances be possible to make use of unstabilised polypeptides, including the native polypeptides of SEQ ID NOS: 13, 14 27 and 34 or the histidine-containing variant of SEQ ID NO: 15 and 33. In the treatment of neurological and cardiac disorders according to the invention, it may be desirable for the polypeptide or extended polypeptide of the invention to be degraded relatively rapidly, i.e. to exert its effect for a relatively short period of time. Therefore, stabilisation will not necessarily be required in the context of such treatments.

Where stabilisation is not required, it is preferred to use the native polypeptides of SEQ ID NOS: 13, 14, 27 and 34 or the Histidine-containing variant of SEQ ID NO: 15 or 33 without stabilising modifications. However, modified polypeptides may also be used. Any of the modifications discussed herein may be applied except that, in this aspect, it is not required that those modifications result in increased stability.

Treatments According to the Invention

Polypeptides and extended polypeptides of the invention can be used to treat a number of conditions. Broadly, these break down into three areas: disorders of skeletal muscle, disorders of cardiac muscle and neurological disorders. However, because nerve and muscle function are inter-dependent, there may be some overlap between these categories, e.g. in the area of neuromuscular disorders.

Neurological disorders may generally be divided into two categories, neurogenic disorders where the fault lies in the nervous system itself and myogenic or muscle-related neurological disorders. Both can be treated according to the invention.

Disorders of skeletal muscle that are susceptible to treatment according to the invention include: muscular dystrophy, including but not limited to Duchenne or Becker muscular dystrophy, Facioscapulohumeral Muscular Dystrophy (FSHD), congenital muscular dystrophy (CMD) and autosomal dystrophies, and related progressive skeletal muscle weakness and wasting; muscle atrophy, including but not limited to disuse atrophy, glucocorticoid-induced atrophy, muscle atrophy in ageing humans and muscle atrophy induced by spinal cord injuries or neuromuscular diseases; cachexia, for example cachexia associated with, cancers, AIDS, Chronic Obstructive Pulmonary Disease (COPD), chronic inflammatory diseases, burns injury etc; muscle weakness, especially in certain muscles such as the urinary sphincter, anal sphincter and pelvic floor muscles; sarcopenia and frailty in the elderly. The invention also finds application in muscle repair following trauma. So far as neurological disorders are concerned, treatment of neurodegenerative disorders is one possibility. Treatment of motoneurone disorders, especially neurodegenerative disorders of motoneurones is also a possibility.

Examples of neurological (including neuromuscular) disorders include amyotrophic lateral sclerosis; spinal muscular atrophy; progressive spinal muscular atrophy; infantile or juvenile muscular atrophy, poliomyelitis or post-polio syndrome; a disorder caused by exposure to a toxin, motoneurone trauma, a motoneurone lesion or nerve damage; an injury that affects motoneurones; and motoneurone loss associated with ageing; and autosomal as well as sex-linked muscular dystrophy; Alzheimer's disease; Parkinson's disease; diabetic neuropathy; peripheral neuropathies; embolic and haemorrhagic stroke; and alcohol-related brain damage. Polypeptides and extended polypeptides of the invention may also be used for maintenance of the central nervous system (CNS). The invention also finds application in nerve repair following trauma.

Nerve damage may also be treated according to the invention. In this embodiment, the polypeptide or extended polypeptide will typically be localised around the sites of such damage to effect repair, e.g. by means of the placement of a conduit around the two ends of a severed peripheral nerve (cf. WO01/85781).

As to cardiac disorders, there may be mentioned diseases where promotion of cardiac muscle protein synthesis is a beneficial treatment, cardiomyopathies; acute heart failure or acute insult including myocarditis or myocardial infarction; pathological heart hypertrophy; and congestive heart failure. Polypeptides and extended polypeptides of the invention may also be used for improving cardiac output by increasing heart stroke volume. In particular, polypeptides and extended polypeptides of the invention may be used for prevention of myocardial damage following ischemia and/or mechanical overload.

In this case, they will generally be administered as rapidly as possible after the onset of the ischemia or mechanical overload to the heart, for example as soon as a heart attack resulting from ischemia has been diagnosed. Preferably, they will be administered within 5, 10, 15, 30 or 60 minutes, or within 2 or 5 hours. Preferably, the ischemia or mechanical overload in response to which the MGF polypeptide or polynucleotide is administered is a temporary condition. In a particularly preferred embodiment, the polypeptide or extended polypeptide of the invention is administered in response to a heart attack. Treatments of the invention will be particularly effective in helping heart attack sufferers make a good recovery; and to return to a normal, active lifestyle.

Under some circumstances, it may be desirable to use polypeptides and extended polypeptides of the invention in combination with other pharmaceutically active agents. For example, polypeptides and extended polypeptides of the invention may be used together with IGF-I (see Examples 1.5, 7 and 8). Such combined uses may involve coadministration of the polypeptides or extended polypeptides of the invention in a single pharmaceutically acceptable carrier or excipient with the other pharmaceutically active agent or agents, or they may involve separate, sequential or simultaneous injection, at the same site or at different sites.

Production of Polypeptides and Extended Polypeptides of the Invention

Polypeptides and extended polypeptides of the invention may be produced by standard techniques. Typically, they will be obtained by standard techniques of peptide synthesis, plus appropriate chemical modifications (e.g. PEGylation) to the resulting amino acid sequence if necessary. Where there are no D-form amino acids, polypeptides and extended polypeptides may instead be obtained via recombinant expression in a host cell from the appropriate coding DNA, again by standard techniques.

Isolation and purification to any desired degree may also be carried out by standard techniques. Polypeptides and extended polypeptides according to the invention will generally be isolated or purified, either completely or partially. A preparation of an isolated polypeptide or extended polypeptide is any preparation that contains the polypeptide or extended polypeptide at a higher concentration than the preparation in which it was produced. In particular, where the polypeptide or extended polypeptide is obtained recombinantly, the polypeptide or extended polypeptide will typically have been extracted from the host cell and the major cellular components removed.

A polypeptide or extended polypeptide in purified form will generally form part of a preparation in which more than 90%, for example up to 95%, up to 98% or up to 99% of the polypeptide material in the preparation is that of the invention.

Isolated and purified preparations will often be aqueous solutions containing the polypeptide or extended polypeptide of the invention. However, the polypeptide or extended polypeptide of the invention may be purified or isolated in other forms, e.g. as crystals or other dry preparations.

Compositions, Formulations, Administration and Dosages

The polypeptides and extended polypeptides of the invention are preferably provided in the form of compositions comprising the polypeptide or extended polypeptide and a carrier. In particular, such a composition may be a pharmaceutical composition comprising the polypeptide or extended polypeptides and a pharmaceutically acceptable carrier or diluent. Any suitable pharmaceutical formulation may be used.

For example, suitable formulations may include aqueous and non-aqueous sterile injection solutions which may contain anti-oxidants, buffers, bacteriostats, bactericidal antibiotics and solutes which render the formulation isotonic with the bodily fluids of the intended recipient; and aqueous and non-aqueous sterile suspensions which may include suspending agents and thickening agents. The formulations may be presented in unit-dose or multi-dose containers. For example, sealed ampoules and vials, and may be stored in a frozen or freeze-dried (lyophilized) condition requiring only the addition of the sterile liquid carrier, for example water for injections, immediately prior to use.

It should be understood that in addition to the ingredients particularly mentioned above the formulations of this invention may include other agents conventional in the art having regard to the type of formulation in question. Sterile, pyrogen-free aqueous and non-aqueous solutions are preferred.

Formulations will generally be tailored, by standard formulation techniques, to the modes of administration discussed below.

The polypeptide of the invention may be administered by any suitable route tailored to the condition to be treated, for example topical, cutaneous, parenteral, intramuscular, subcutaneous or transdermal administration; or by direct injection into the bloodstream or direct application to mucosal tissues.

Injection is likely to be the preferred route under many circumstances, for example subcutaneous, parenteral intramuscular or intravenous injection. Intravenous injection will often be preferred under many clinical circumstances. So-called “needle-less” injection or transcutaneous administration may be possible under some circumstances.

In the treatment of skeletal muscle disorders, intravenous and intramuscular injection are preferred routes. Topical administration is also envisaged, e.g. via patches, to strengthen the muscles of the abdomen or for other purposes.

In the treatment of cardiac muscle disorders, delivery will generally be intravenous. Under appropriate clinical circumstances (e.g. in specialist cardiac units) direct delivery to the heart may also be possible, e.g. using a so-called “needle-less” injection system for delivery the polypeptide to the heart.

The polypeptides and extended polypeptides of the invention may be delivered in any suitable dosage, and using any suitable dosage regime. Persons of skill in the art will appreciate that the dosage amount and regime may be adapted to ensure optimal treatment of the particular condition to be treated, depending on numerous factors. Some such factors may be the age, sex and clinical condition of the subject to be treated.

Based on the Inventors' experience, it is envisaged that doses in the region of 0.2 to 10 mg will be effective, for example 0.2 to 0.8 mg, preferably about 0.5 mg. For example, a solution containing the polypeptide or extended polypeptide at a concentration of 1 mg/ml may be used in an amount of 0.1 to 1 ml. Single or multiple doses may be given, depending on the application in question and the clinical circumstances.

The following Examples illustrate the invention.

### EXAMPLES

1. Peptides

1.1 Peptides of Examples 2, 3, 4 and 6

The peptide used in Examples 2, 3, 4 and 6 had the sequence of SEQ ID NO: 15), in which the penultimate Arginine of the native sequence (See SEQ ID NOS: 1, 2 and 27) is replaced by Histidine, stabilised by the use of the D form of Arginine instead of the naturally occurring L-form at positions 14 and 15 and the covalent attachment of the N-terminus to a polyethylene glycol (PEG) derivative (O′O-bis(2aminopropyl)polyethylene glyclol 1900) (Jeffamine) via a succinic acid bridge, and amidated at the C-terminus.

1.2 Peptides of Example 5

The peptides of Example 5 were obtained from Alta Biosciences, Birmingham, UK, having been synthesised via standard techniques using a peptide synthesiser. These peptides are unPEGylated and free from L-D conversion and C-terminal amidation.

Also, a peptide corresponding to that of 1.1 above, with the same L-D conversions, but without PEGylation, has also been tested for stability (see 5.2.3 below). This peptide was synthesised via standard techniques using a peptide synthesiser. The product was purified by HPLC and analyzed by MALDI-MS.

1.3 Peptides of Example 7

1.3.1 Peptides of Example 7.1

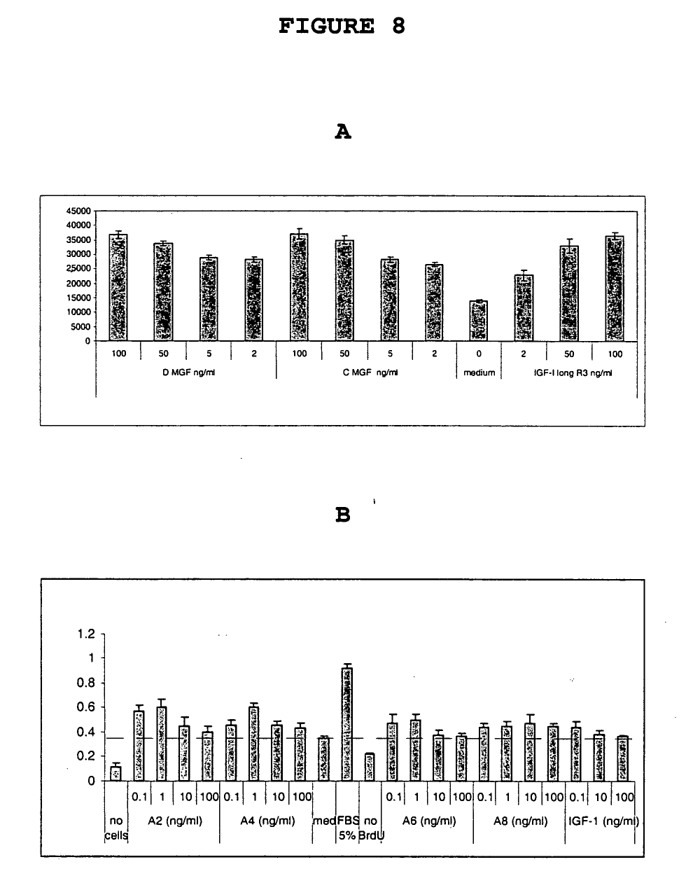

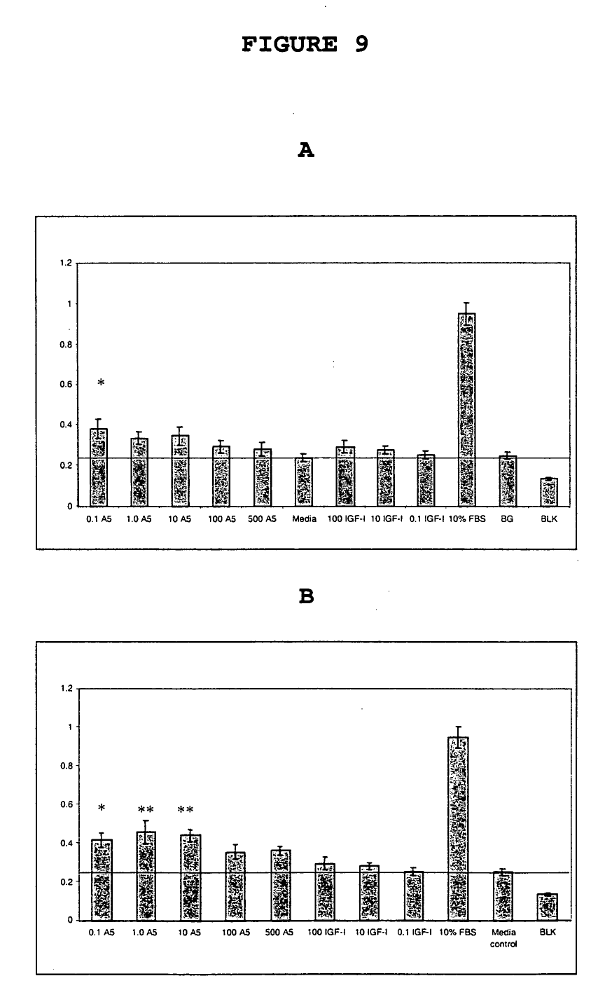

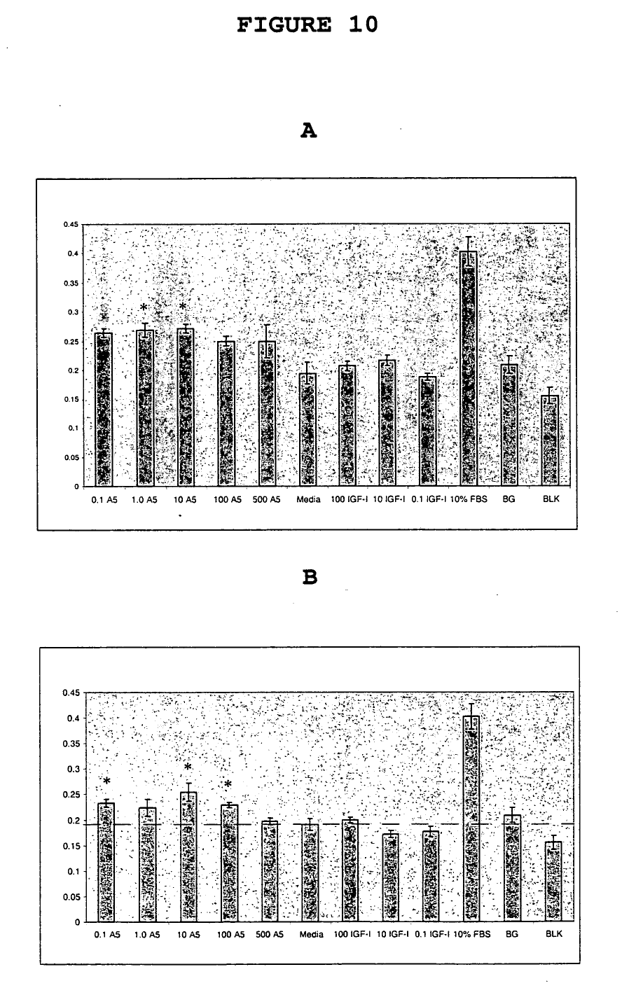

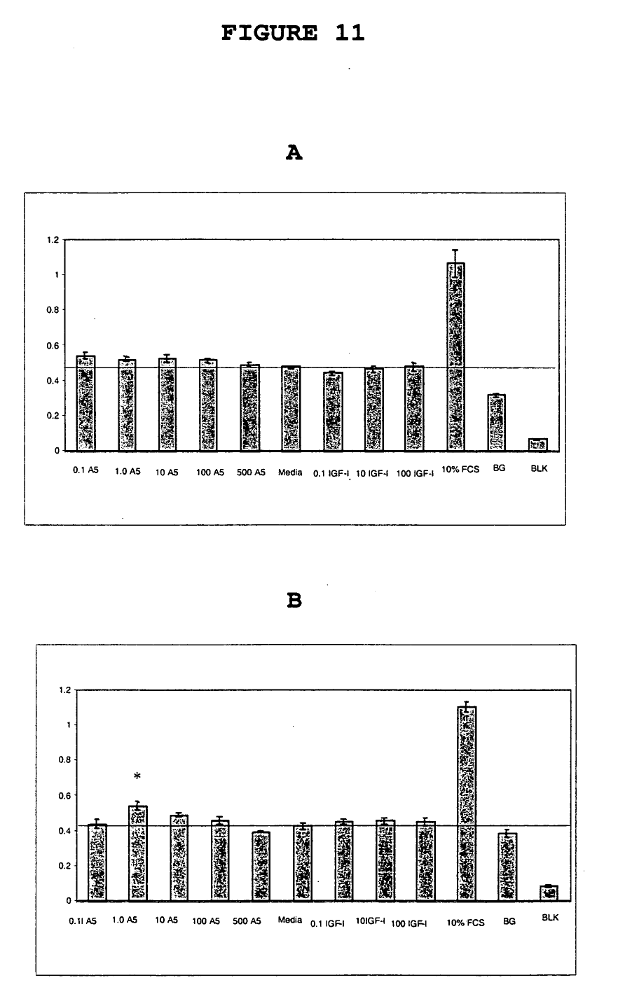

The peptides of Example 7.1 had the 8 amino acid sequence Gly-Ser-Thr-Phe-Glu-Glu-His-Lys (SEQ ID NO:33), plus modifications to improve stability. In the peptide referred to as DMGF in FIG. 8, stabilisation was achieved via N-terminal PEGylation as in 1.1 above. In the peptide referred to as CMGF in FIG. 8, stabilisation was achieved via N-terminal attachment of hexanoic acid acid. Both DMGF and CMGF were also amidated at the C-terminal end.

1.3.2 Peptides of Example 7.2

In Example 7.2, peptides A2, A4 and A6 had the sequence Gly-Ser-Thr-Phe-Glu-Glu-Arg-Lys (SEQ ID NO:34). Peptides A2, A4 and A6 were amidated at the C-terminus.

Peptide A2 was unmodified at the N-terminus. Peptide A4 had a hexanoic acid moiety attached at the N-terminus. Peptide A6 had an amino-hexanoic acid moiety attached at the N-terminus.

Peptide A8 had the sequence Gly-Ser-Thr-Phe-Glu-Glu-His-Lys (SEQ ID NO:33), amidated at the C-terminus and with hexanoic acid attached at the N-terminus.

1.4 Peptide of Example 8

The peptide used in Example 8 had the sequence of SEQ ID NO: 15, in which the penultimate Arginine of the native sequence (See SEQ ID NOS: 1, 2 and 27) is replaced by Histidine, stabilised by the use of the D form of Arginine instead of the naturally occurring L-form at positions 14 and 15 and amidated at the C-terminus. The peptide used in Example 8 was not pegylated.

1.5 IGF-I Peptide

For comparison, IGF-I peptide has been used. This is the IGF-I receptor binding domain encoded by Exons 3 and 4 that is common to all splice variants and is approximately 70 amino acids in length. In Examples 1-4, this was obtained from PeproTech, EC, UK. In Example 6, it was obtained from Sigma-Aldrich (ER2 IGF-I). IGF-I peptide was also used in Example 7.

2. Injection of Stabilised Peptide into Dystrophic Muscle

Using intramuscular injections (twice weekly injections of 25 μl containing 17 μg of the chemically stabilised peptide), muscle strength was increased by more than 25% within a few weeks in the tibialis anterior muscle of non-dystrophic mice This muscle is not diseased like the muscle of mdx mice (see below), although it is possible that it was physically damaged by the repeated injections.

Greater increases, of up to around 35% (FIGS. 3A, 3B), were recorded for intramuscular injections (two per week for three weeks) in the dystrophic muscles of the mdx mouse, which has the same type of mutation as that in human Duchenne muscular dystrophy. Injections of IGF-I led only to an increase of around 5%, as shown in FIG. 3A On the same basis, the results of a comparison between the stabilised peptide and a PBS vehicle-only control are shown in FIG. 3B.

These data relating to muscle protection and repair show that the stabilised peptide is effective in increasing the strength of dystrophic and non-dystrophic muscle.

3. Cardioprotection and Myocardial Repair by Stabilised Peptide

A myocardial infarction (MI) was induced in ovine hearts by catheterising a marginal branch of the circumflex coronary artery and injecting a small bolus of microspheres to induce localised ischemia. Full-length MGF (native C-terminal peptide plus sequence encoded by exons 3 and 4 and common to MGF and liver-type IGF-I) or stabilised peptide was injected (200 nm, intracoronary) 15 minutes later using the same catheter whilst the animal was still under the anaesthetic. As a control, mature liver-type IGF-I was used. The use of the stabilised peptide alone was found to markedly increase the percentage of viable myocardium and the ejection fraction as measured by echocardiography and computerised analysis of the ejection function following the MI. Full-length MGF also had a significant, though smaller effect.

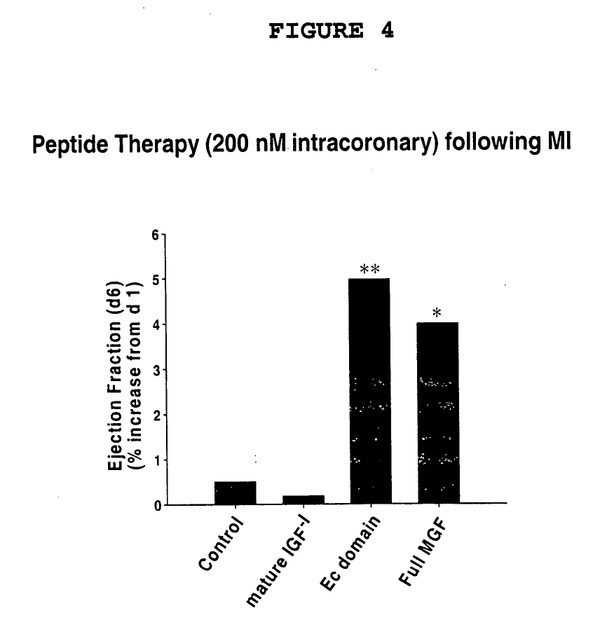

Mature liver-type IGF-I had a much smaller effect. The results are given in FIG. 4, which shows percentage change in ejection fraction on day 6 as compared to ejection fraction on day 1 before the procedure was carried out. Thus, the stabilised peptide was very effective in protecting the myocardium from ischemic damage.

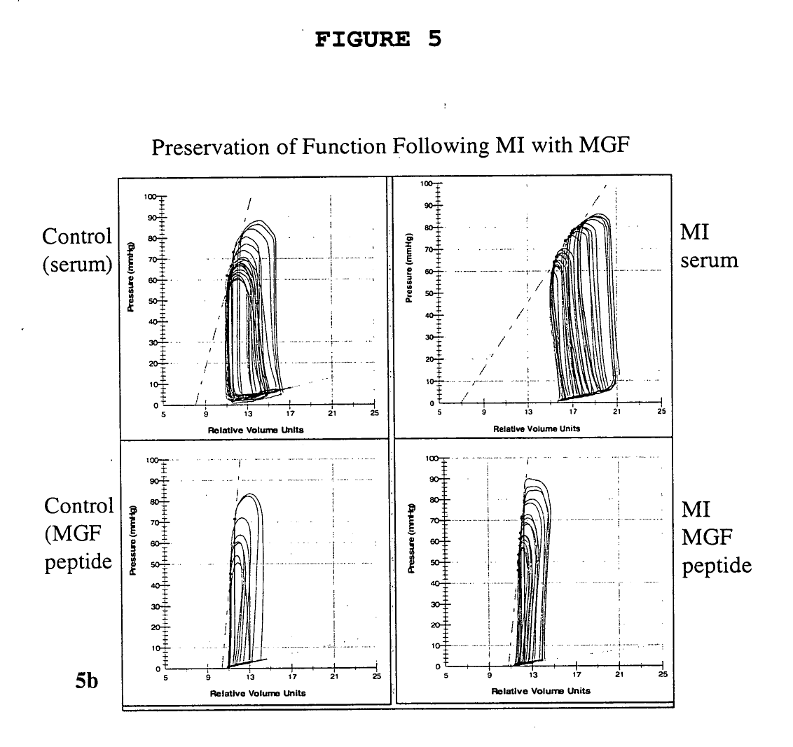

Additional experiments were carried out on mice. In these studies the MI was produced by ligating the left anterior descending (LAD) coronary artery of the murine heart. This causes dilation of the left ventricle, the progression of which leads to heart failure. Stabilised peptide administered systemically markedly improved the strength and function of the heart as measured by the pressure/volume loops (FIG. 5) that demonstrate the ability of the heart to pump blood and the dilation that results when the damaged heart can no longer cope with the venous return. This is markedly improved by the systemic administration of the stabilised peptide, through which the myocardial wall muscle is protected and increased in thickness. Therefore there is considerable potential for treatment of patients immediately following a heart attack.

4. Neuroprotection by Stabilised Peptide Following Ischemia and General Damage

4.1 Neuroprotective Effect In Vitro

The neuroprotective effect of the stabilised peptide was demonstrated in vitro using the well-characterised model of selective neuronal death in rat organotypic hippocampal cultures.

Hippocampal slices were prepared from 7-10 days old Wistar rats according to the method of Stoppini et al (1991) with minor modifications according to Sarnowska (2002). Briefly, rats were anaesthetised with Vetbutal, ice-cooled and decapitated. Brains were quickly removed to ice-cold working solution pH 7.2: 96% of HBSS/HEPES-(Ca2⁺ and Mg2⁺ free) containing 2 mmol/L L-glutamine, 5 mg/ml glucose, 1% amphotericine B, 0.4% penicillin-streptomycin. Hippocampi were separated and cut into 400 μm slices using McIlwain tissue chopper. Millicell-CM membranes (Millipore) in 6-well plates were pre-equilibrated with 1 ml of culture medium pH 7.2: 50% DMEM, 25% HBSS/HEPES, 25% HS, 2 mmol/L L-glutamine, mg/ml glucose, 1% amphotericine B, 0.4% penicillin-streptamycine in a moist atmosphere of air and 5% CO₂ at 32° C. for 30 minutes. Four selected slices were settled on each membrane. Slices were cultivated for two weeks at 32° C. in 5% CO₂ atmosphere of 100% humidity. The viability of the slices was checked daily under the light microscopy and evaluated additionally on the day of experiment by propidium iodide staining and observed under fluorescent microscope (Zeiss Axiovert 25) with MC-10095 camera (Carl Zeiss Jena GmbH) in order to record initial PI uptake (Sarnowska, 2002).

Oxidative stress was induced after 14 days in culture by adding 30 mM TBH (tert-butyl peroxide) for 3 hours. After that time the slices were transferred to the fresh culture medium. Resulting cell death was assessed 24 and 48 h after the beginning of the experiment.

Stabilised peptide or, for the purpose of comparison, recombinant IGF-1 was added to the culture medium to the final concentration of 100 ng/ml at the beginning of the experiment and was continuously present in the medium.

In order to investigate a pathway in which the MGF acts, a specific anti-IGF-1 receptor (AB-1) blocking antibody (Oncogene) was included in the medium 1 hour before the slices were exposed to TBH and MGF or IGF-1 peptide. The concentration of the antibody (1000 ng/ml) was used according to the manufacturer's recommendation.

To obtain detailed images of the slices, a confocal laser scanning microscope (Zeiss LSM 510) was used. A helium-neon laser (543 nm) was used for the excitation of propidium iodide (PI). Following acquisition, images were processed using the Zeiss LSM 510 software package v. 2.8. Quantitative measurement of tissue deterioration was performed using image analyser KS 300 (Carl Zeiss Jena GmbH).

Cell damage was quantified on fluorescence images of PI-stained cultures 24 and 48 hours after TBH challenge. The relative extent of cell death was calculated from each standardized CA1 region as follows: % of dead cells=(experimental fluorescent intensity (FI)−background FI)/(maximal FI−background FI)×100, where maximal FI was obtained by killing all cells with exposure to 100 mM glutamate.

All the measurements were repeated for 5 independent culture preparations and 8 slices were used for each experimental condition. Statistical significance of the differences between the results was calculated using one-way Anova followed by Dunnet's test, (GraphPad Prism 3.02).

Rat brain slices were isolated following induction of localized damage by TBH (tert-butyl hydroperoxide) as discussed above. The resulting cell death in treated and non-treated brain slices was determined. This is illustrated in FIG. 6. In the absence of treatment peptide, TBH caused about 60% of the cells to die within 24 hours but, following treatment with the stabilised peptide (10 ng/ml), 85% protection was observed. The IGF-I receptor domain peptide (rIGF-I), which is also part of full length MGF, was also neuroprotective (as previously reported). However, this was to a lesser degree (72%) and the protective effect of IGF-1 was only noticeable for up to 24 hours, whereas the stabilised peptide functioned for significantly longer as its neuroprotective effect was still clearly observed after 48 hours.

4.2 Neuroprotective Effect in Gerbil Model

Other experiments were carried out using a Gerbil model of brain ischemia. To assess neuroprotection, confocal microscopy was carried out on the brain after administration of the stabilised peptide or the IGF-I receptor binding domain.

In the gerbil brain, bilateral ligation of the common carotid arteries invariably produces specific hippocampal lesions: in the CA1 region, pyramidal neurones start to die 3-4 days after ischemia.

Male Mongolian gerbils weighing 50-60 g were used. The ischemic insult was performed by 5 min. ligation of the common carotid arteries under halotane in N₂O:O₂ (70:30) anaesthesia in strictly controlled normothermic conditions as previously described (Domańiska-Janik et al., 2004). The cerebral blood flow was continuously monitored by laser Doppler flowmetry (Muro, Inc.). A group of animals received stabilised peptide or IGF-1 (1 μg/μl in PBS) by injection at a dose of 25 μg directly to the left carotid artery immediately upon the reperfusion. Sham operated animals were injected with the same dose of the peptide.

Usually, 10-15 minutes after the procedure, treated animals were standing up on their legs and behaving as untreated ones. The animals were allowed a recovery period of one week, then were perfused with ice-cold 4% paraformaldehyde in PBS under pentobarbital anaesthesia. The histological evaluation was performed on paraffin-embedded and fixed, 10 mm-thick sections stained by hematoxylline/eosine. The extent of cell damage in the CA1 hippocampal region was quantified, under a Zeiss Axioscop 2, as the mean number of the persisted, intact neurons in the coronal sections. At least three defined 300 μm fields of the CA1 region were captured using a MC 10095 camera (Carl Zeiss Jena GmbH) and counted in a computer-assisted image analysis system (KS 300, Carl Zeiss Jena GmbH).

In control animals, the mean number of morphologically intact neurones per 300 μm length scored in the CA1 region was 121.25±12.5 (mean±SD, n=5). In contrast to the untreated animals, where only about 12% (15.2±5, n=7) of neurones survived the ischemic episode, injection (single bolus of 25 μg) of the stabilised MGF C-terminal peptide into the left carotid artery, immediately after re-perfusion, provided a very significant neuroprotection. 83.2±25 (n=10) neurones were scored on the injected side (74.5% of non-operated control value) and 65.8±30 (n=10) on the contralateral side (54% of non-operated control value). Thus, treatment with the stabilised MGF C-terminal peptide enabled a high proportion of the CA1 hippocampal neurones to survive the ischemic insult. In most animals, the protective effect was noticeable bilaterally while in a minority it was mostly evident on the injected (left) side.

In contrast, similar injection of 25 μg of IGF-1 peptide had little influence on the postischemic survival of CA1 neurones; 7 days after the insult there were 19.2±7.3 neurones (n=5) left, which is only 15.8% of the control neuronal cell number and not significantly different from the untreated postischemic group.

5. Biological Activity and Stability of Modified Peptides

5.1 Peptide Stabilised by L-D Conversion and N-terminal PEGylation

The peptide used in Examples 2, 3 and 4 above had the sequence of SEQ ID NO: 15 (which corresponds to that of the the human Ec peptide of MGF (SEQ ID NO: 27), except that Arginine in the penultimate position is replaced by Histidine) stabilised by the use of the D form of Arginine at positions 14 and 15 instead of the naturally occurring L-form and the covalent attachment of the N-terminus to polyethylene glycol (PEG), and amidated at the C-terminus.

The biological activity of this peptide is confirmed in Examples 2, 3 and 4.

Its stability is demonstrated by FIG. 7. Stability of the peptides with and without PEGylation and L-D conversion of Arginine at positions 14 and 15 was investigated by assessing the peptides' susceptibility to proteolytic cleavage in fresh human plasma.

The plasma was stored until used at −70° C. 10 μg of peptide were added to 2 ml of plasma, plus 7 ml of PBS. This mixture was incubated at 37° C. for different time intervals. Western blotting with a polyclonal antibody having specificity to peptides with the amino acid sequence of SEQ ID NO: 15 was then used to detect each peptide over those time intervals. (In FIG. 7: A=0 minutes; B=30 minutes; C=2 hours; D=24 hours. The results for the peptide with L-D conversion and N-terminal PEGylation are shown on the right; those for the peptide lacking the L to D form conversions and N-terminal PEGylation are on the left.). Relatively little of the peptide lacking L-D conversion and PEGylation could be detected after 30 minutes, very little after 2 hours and none or almost none after 24 hours. In contrast, the peptide with L-D conversion and PEGylation could be detected in abundance even after 24 hours.

5.2 Further Peptides—Replacement of Serine or Arginine with Alanine and C-Terminal and N-Terminal Truncation

5.2.1 Further Peptides

Herein, the sequence of the native human Ec peptide from the C-terminus of human MGF is given as SEQ ID NO: 27. In the peptide of SEQ ID NO: 15, the penultimate amino acid, which is Arginine in the native peptide (See SEQ ID NOS 2 and 27) is replaced with Histidine. The peptide of SEQ ID NO: 15 is described as Peptide 1 in FIG. 2.

Further modified sequences derived from the sequence of SEQ ID NO: 15 are given as SEQ ID NOS: 16 to 24 and compared to that of SEQ ID NO: 15 in FIG. 2, where they are referred to as Peptides 2-6 and Short peptides 1-4.

In Peptide 2 (SEQ ID NO: 16), Serine is replaced with Alanine at position 5. In Peptide 3 (SEQ ID NO: 17), Serine is replaced with Alanine at position 12. In Peptide 4 (SEQ ID NO: 18), Serine is replaced with Alanine at position 18. In Peptide 5 (SEQ ID NO: 19), Arginine is replaced with Alanine at position 14. In Peptide 6 (SEQ ID NO: 20), Arginine is replaced with Alanine at position 14 and Arginine is also replaced with Alanine at position 15. In Short peptide 1 (SEQ ID NO: 21), Arginine is replaced with Alanine at position 14 and the two C-terminal amino acids are removed. In Short peptide 2 (SEQ ID NO: 22), Arginine is replaced with Alanine at position 14 and the four C-terminal amino acids are removed. In Short peptide 3 (SEQ ID NO: 23), Arginine is replaced with Alanine at position 14 and the three N-terminal amino acids are removed. In Short peptide 4 (SEQ ID NO: 24), Arginine is replaced with Alanine at position 14 and the five N-terminal amino acids are removed.

5.2.2 Biological Activity of Further Peptides

Biological activity was determined using an in vitro system by measuring the ability of the C terminal peptides to induce mononucleated myoblasts (satellite cells) to replicate. Cell number was determined using the Alamar Blue method. This was assessed on a scale of 0 to 3 and the results are shown in FIG. 2.

0=no measureable increase in cell number at 6 h.

1=significant increase in cell number at 4 h.

2=significant increase in cell number at 2 h.

3=significant increase in cell number at 1 h.

Significance was at the level of P>0.05 using the T test.

The peptide (Peptide 1) of SEQ ID NO: 15 showed little or no activity owing to its short half-life. Peptide 2 (SEQ ID NO: 16) and Short Peptide 1 (SEQ ID NO: 21) scored 1 on the activity scale. Peptides 4 and 5 (SEQ ID NOS: 18 and 19) scored 2 on the activity scale. Peptide 3 (SEQ ID NO: 17) scored 3 on the activity scale. Peptide 6 (SEQ ID NO: 20) and Short peptides 2, 3 and 4 (SEQ ID NOS: 22, 23 and 24) exhibited no measurable activity (zero score).

5.2.3 Stability of Further Peptides

The stability of each peptide was determined by introducing it into fresh human plasma and using Western blotting as discussed in Example 5.1 above. Like biological activity, stability was scored on a scale of 0 to 3. The results are shown in FIG. 2.

Stability was determined as the amount of the peptide that remained intact and bound to the specific antibody in the following way:

1=marked loss of detectable antibody binding by ½ hours.

2=marked loss of detectable binding by 2 hours.

3=no marked loss of antibody binding by 24 hours.

The peptide (Peptide 1) of SEQ ID NO: 15 scored 1. Peptide 6 (SEQ ID NO: 20) also scored 1. Peptides 3 and 4 (SEQ ID NOS: 17 and 18) scored 2. Peptides 2 and 5 (SEQ ID NOS: 16 and 19) scored 3.

The peptide of Examples 1-4 also scored 3 on this scale. The same peptide, but lacking PEGylation, also scored 3 on this scale. Short peptides 1-4 have not yet been tested, though Short peptides 2 to 4 appear to lack biological activity anyway.

6. Effects of Stabilised Peptide on Muscle Satellite Cell Proliferation in Dystrophic, ALS and Healthy Human Muscle

The stabilised peptide of 1.1 above was used in these experiments. Comparisons were made with the IGF-I peptide of 1.3 above.

6.1 Summary

Primary human muscle cell cultures were derived from biopsies of congenital muscular dystrophy (CMD), facioscapulohumeral dystrophy (FSHD) and motor neurone disease or amyotrophic lateral sclerosis (ALS) patients as well as from healthy muscle using proliferation/differentiation assays. Cell cultures were treated with the two peptides and immunocytochemistry techniques were used to detect cells expressing the differentiation marker desmin, and total number of nuclei using DAPI. Creatine phosphokinase (CPK) and protein assays were used to determine myogenic differentiation following peptide treatment. The stabilised peptide considerably increased stem (desmin positive) cell proliferation for normal (non-diseased) muscle (from 38.4±2.5% to 57.9±3.2% in normal (non-diseased) limb and from 49.8±2.4% to 68.8±3.9% for normal (non-diseased) craniofacial muscle biopsies). Although the initial muscle stem cell numbers were lower in patients with muscle wasting, the stabilised peptide still induced an increase (CMD 10.4±1.7% to 17.5±1.6%; FSHD 11.7±1.3% to 20.4±2.1% and ALS 4.8±1.1% to 7.2±0.8%). The results also confirmed that the stabilised peptide had no effect on myotube formation but that it increases myoblast progenitor cell proliferation, whilst mature IGF-I enhanced differentiation.

6.2 Isolation of Human Muscle-Derived Cells

Human primary muscle cell cultures were isolated as previously described [Lewis et al., 2000; Sinanan et al., 2004]. Briefly, following informed consent, craniofacial (masseter) muscle biopsies were obtained from healthy adult and CMD patients during elective surgery at the Eastman and Middlesex Hospitals, London, UK. Human lower limb (vastus lateralis) muscle samples were obtained from consenting, adult healthy, FSHD and ALS patients by needle biopsy under local anaesthesia at the Royal Free Hospital, London, UK. Biopsies were pooled from several patients with the same disorder to obtain sufficient cell numbers in the primary cultures. These were washed with antibiotic (penicillin, 100 U/ml; streptomycin, 100 μg/ml; fungizone, 2.5 μg/ml; Invitrogen) supplemented DMEM (high glucose; Invitrogen), scissor-minced and tissue fragments plated into 0.2% gelatin-coated (Sigma-Aldrich) T150 cm² culture flasks (Helena Biosciences). Explant cultures were incubated in serum-containing Growth Media (sGM), composed of DMEM, 20% FCS (PAA Laboratories), penicillin (100 U/ml) and streptomycin (100 g/ml) (Invitrogen), and maintained at 37° C. in humidified 95% air with 5% CO₂. The first wave of migration of mononuclear cells from the explant was designated the □-wave and this population was used throughout this study. Migratory human muscle cell were enzymatically harvested using trypsin-EDTA (Invitrogen) and subcultured in sGM until 70-80% confluency. Passage number x (Pₓ), was defined as the xth sequential harvest of subconfluent cells. All experiments were performed using P₃₋₅ cohorts. The expanded cells were then stored under cryogenic conditions until they were used in the experiments described below. At least 6 runs were made for each of the treatments used for each diseased muscle culture as well as for the two types of healthy muscle.

6.3 Determination of the Myogenic Progenitor (Stem) Cell Population In Vitro

Assessment of the number of myogenic precursors was performed as described previously (Sinanan et al., 2004). Cells were re-plated on gelatin-coated (0.2%) 13 mm coverslips at an initial density of 4.5×10³ cells cm⁻². To avoid confounding effects of IGF and related protein in FCS, cells were cultured in a serum-free, defined media (dGM); DMEM supplemented with EGF (10 ng/ml), bFGF (2 ng/ml), insulin (5 ng/ml), holo-transferrin (5 μg/ml), sodium selenite (5 ng/ml), dexamethasone (390 ng/ml), vitamin C (50 μg/ml), vitamin H (D-biotin; 250 ng/ml), Vitamin E (Trolox; 25 μg/ml) (Sigma-Aldrich), albumax-1 (0.5 mg/ml) (Invitrogen), fetuin (500 μg/ml) (Clonetics/BioWhittaker), penicillin (100 U/ml) and streptomycin (100 μg/ml) (Invitrogen). After allowing 24 hours for adherence, the stabilised peptide (10 ng/ml) with and without rIGF-I, (10 ng/ml) and with and without monoclonal IGF-I receptor antibody (Ab-1, 100 μg/ml, Oncogene) were added in dGM as appropriate. The peptides used were (see 1.1 and 1.3 above) the stabilised peptide related to the E domain of MGF/IGF-IEc peptide [24 amino acid residues] synthesized as described previously [Dluzniewska et al, 2005] and human IGF-I peptide [70 amino acid residues] (Sigma-Aldrich ER2 IGF-I). All media were replaced every 2-3 days. The cultures were sampled at various time-points for immunocytochemical analyses.

6.4 Immunocytochemistry

At the appropriate time-points, cells were fixed with methanol for 10 min (−20° C.), followed by detergent permeabilization with 0.5% Triton-X100 for 10-15 min. Cells were then incubated for 60 min with an anti-desmin (1:100; clone D33, DAKO, Glostrup, Denmark) antibody, diluted in antibody diluting solution (ADS; PBS plus10% FCS, 0.025% sodium azide, 0.1M lysine). A class specific anti-mouse IgG antibody conjugated to FITC (1:200; Jackson ImmunoResearch Laboratories/Stratech Scientific) was used to visualize. Nuclei were identified by introducing the fluorescent minor-groove DNA-binding probe, DAPI (1.0 ng/ml; Sigma-Aldrich), into the final antibody incubation step. Coverslips were mounted with the glycerol-based anti-fade agent, Citifluor (Citifluor Ltd), and sealed with clear nail varnish. Cell-associated fluorescence and morphology, were visualized by epi-fluorescence and Leica Modulation Contrast (LMC) microscopy respectively, using an inverted Leica DMIRB microscope equipped with Leica FW4000 image processing software. For the proliferation assay, all blue and green fluorescent positive cells were counted in a field. At least 30 fields in each coverslip were counted in a systematic manner; at least 100 cells were therefore counted on each coverslip. The number of cells was compared as the percentage of desmin positive cells to the total number of DAPI positive cells.

6.5 Creatine Phosphokinase (CPK) Assay

This assay was performed using previously published protocols [Auluck et al., 2005]. Measurement of CPK allows for the quantitative comparison of myogenesis [Goto et al., 1999], as it is a marker of myotube formation. The enzyme CPK catalyzes the reversible phosphorylation of adenosine-5-diphosphate (ADP) to form adenosine-5-triphosphate (ATP) and free creatine. The reaction may be followed in either direction by measuring the formation of inorganic phosphorus, an end-product of the reaction which is proportional to CPK activity. This was measured using the calorimetric method based on the generation of inorganic phosphate [Fiske and Subbarow, 1925] procedure. This was then expressed in terms of the protein content of the culture.

Previously expanded primary human muscle cell cultures were re-plated at 10×10⁴ cells/well in 0.2% gelatin coated 96 well plates. Cells were cultured until 70/80% confluent in sGM then the medium changed to differentiation medium (DM; DMEM, 2% FCS, penicillin (100 U/ml) and streptomycin (1001 g/ml)) containing the stabilised peptide [24 amino acid residues] synthesized as previously described [Dluzniewska et al, 2005] and/or human IGF-I peptide [70 amino acid residues] (Sigma-Aldrich IGF-I ER2). After 48 hours, cells were washed twice with ice cold PBS and then stored frozen in 0.5 mM glycine buffer (pH 6.75) at −70° C. Fixed cells were lysed by rapid thawing and CPK assay kit used according to manufacturers instructions (Sigma-Aldrich). The protein concentration of each sample was determined against an albumin standard curve using the Pierce Micro BCA Kit (PerBio Science, UK Ltd., Northumberland, UK).

6.6 Statistical Analysis

1-way ANOVA test was applied using StatView 4.51 (SAS Institute Inc., Cherwell Scientific Publishing Ltd, Oxford, UK) followed by the Fisher's PLSD post hoc test. p<0.05 was considered significant. Data were pooled for all runs (minimum of 6) for the 4 types of experiments for each condition including the two types of healthy muscle and presented as mean±s.d.

6.7 The Proportion of Myogenic Precursors in Human Muscle Primary Cultures

The percentage of myogenic (desmin positive) cells was determined from all of the muscles tested (See Table below). Normal (non-diseased) muscle contained a significant proportion of desmin positive cells whereas diseased muscle contained a much lower proportion of myogenic cells.

TABLE
Human primary muscle cultures derived from different muscle sources
that contain differing proportions of myogenic (desmin positive) cells
before addition of peptides

| | Desmin positive cells as percentage |
|---|---|
| Muscle Type | of total cells in primary culture. |
| Normal (non-diseased) | 49.8 ± 2.4% |
| Craniofacial | |
| Normal (non-diseased) Limb | 38.4 ± 2.5% |
| CMD Limb | 10.4 ± 1.7% |
| ALS Limb | 4.8 ± 1.1% |
| FSHD Limb | 11.7 ± 1.3% |

6.8 Effect on Normal (Non-Diseased) Human Primary Muscle Progenitor Cells

The stabilised peptide increased proliferation (changes in the proportion of desmin-associated nuclei to total nuclei) significantly in normal craniofacial (masseter) primary cultures from 49.8±2.4% to 68.8±3.9%; p<0.0001). IGF-I also induced a moderate increase (from 49.8±2.4% to 58.4±4.2%; p<0.0001). Interestingly, it was found that the effect of the stabilised peptide on desmin positive cell proliferation ratio was inhibited when IGF-I was added (from 68.8±3.9% to 59.5±4.2%; p<0.0001). The effect seen in normal lower limb (quadriceps) primary cultures was similar to that seen with craniofacial muscle. The stabilised peptide increased muscle progenitor cell proliferation significantly (from 38.4±2.5% to 57.9±3.2%; p<0.0001). IGF-I had only a minor effect on proliferation (from 38.4±2.5% to 47.1±3.5%; p<0.0001) but IGF-I completely abrogated the response to the stabilised peptide when the two peptides were added in combination (from 57.9±3.2% to 38.8±0.6%; p<0.000).

6.9 Effect on Disease-State Human Primary Muscle Derived Cell Proliferation

Following the observation that the stabilised peptide could reproducibly and significantly increase the number of desmin positive cells in normal muscle, the effect on disease-state muscle was investigated. In primary cultures derived from congenital muscular dystrophy (CMD), the stabilised peptide significantly increased muscle progenitor cell proliferation (from 10.4±1.7% to 17.5±1.6%; p<0.0001), whilst IGF-I had a small effect (10.4±1.7% to 13.2±1.7%; p=0.005). When combining both peptides the inhibiting effect was again as observed as for normal muscle, with effect of the stabilised peptide being reduced to control levels (from 17.5±1.6% to 13.1±1.2%; p=0.0001). The effects of the stabilised peptide on cellular proliferation of muscle cells from amyotrophic lateral sclerosis—(ALS) and FSHD (produced similar results. The stabilised peptide increased the numbers of desmin expressing cells markedly in these disorders (ALS from 4.8±1.1% to 7.2±0.8%; p=0.0002, FSHD from 11.7±1.3% to 20.4±2.1%; p<0.0001). As was the case for normal muscle, IGF-I again had a negligible effect (ALS from 4.8±±1.1% to 4.7±1.4%; p=0.7719, FSHD from 11.7±1.3% to 14.1±1.6%; p=0.0107)). When both isoforms were used together, MGF-induced desmin expressing increase was again inhibited (ALS from 7.2±0.8% to 5.3±1.0%; p=0.0024, FSHD from 20.4±2.1% to 14.5±1.4%; p<0.0001).

6.10 Increased Progenitor Cell Proliferation by MGF E Domain in Relation to the IGF-I Receptor

In normal muscle, the increase in proliferation induced by the stabilised peptide was not inhibited by the presence of an anti-IGFR antibody (68.8±3.9% in MGF treated and 71.1±6.2% in MGF plus Ab-I treated cells; p=0.2472). The same effect was also observed for both CMD and ALS muscle (17.5±1.6% vs. 16.7±1.8% p=0.4589 for CMD; 7.2±0.8% vs. 6.5±0.8%, p=0.2933 for ALS). This indicates that the action of the MGF E domain does not involve the IGF-I receptor.

6.11 Effects of MGF E Domain on Preventing Terminal Differentiation.

In the CPK assays of 6.4 above, the stabilised peptide did not facilitate primary myoblast differentiation and myotube formation. In contrast, IGF-I at a concentration of 10 ng/ml apparently stimulates myotube formation as the numbers of cells expressing desmin is decreased by the addition of IGF-I on this stage of myogenesis. Indeed, in the presence of 10 ng/ml IGF-I, the stabilised peptide acted as an agonist and, in a dose-dependent manner, prevented differentiation to the myoblast fusion competent stage. The decrease of 100 ng/ml of the stabilised peptide with 10 ng/ml of systemic IGF-I was lower than 10 ng/ml of MGF with the same dose of IGF-I.

6.12 Conclusions

The stabilised peptide induced progenitor cell proliferation significantly in primary muscle culture from patients with CMD, FSHD and ALS as well as healthy individuals. The stabilised peptide did not affect myotube formation, a process that IGF-I accelerates significantly. This demonstrates that the biologically active MGF E domain has a distinct activity compared to mature IGF-I. Our findings indicate that the different actions of IGF-I isoforms are probably mediated via different receptors. The blocking of the IGF-I receptor provides evidence that MGF E domain increases satellite cell proliferation via a different signalling pathway to IGF-I, and that the initial satellite cell activation is a separate process from that which is influenced by mature IGF-I.

It has been proposed that muscle wasting in neurological conditions and ageing is due to a loss of satellite cells. We have demonstrated that the ratio of progenitor (desmin positive) cells to total myoblasts from the patients with CMD, FSHD and ALS is low compared to the ratio of myoblasts from healthy individuals. Thus it is debatable whether muscles degenerate because of lack of satellite cells or because of inability to express some factor for satellite cell activation. We have previously demonstrated that elderly people are unable to express MGF at the levels required to maintain muscle [Hameed et al., 2004], with similar findings for FSHD and ALS patients (unpublished findings).

Muscle wasting is one of the main causes of death in patients with certain neuromuscular diseases. Muscle loss can be linked to the inability to express MGF, and that muscles of the mdx dystrophic mouse, a model for human Duchenne Muscular Dystrophy, are unable to produce MGF even during mechanical stimuli [Goldspink et al., 1996]. De Bari et al found that when mesenchymal stem cells were introduced into dystrophic muscles of mdx mouse, the sarcolemmal expression of dystrophin and also MGF expression was restored [De Bari et al., 2003]. Therefore, the production of MGF may depend on the compliance of the cell membrane and possibly involve some type of mechanotransduction mechanism e.g. the dystrophin complex

It has been known for some time that IGF-I is a neurotrophic factor, and possesses potential clinical applications for neurodegenerative disorders, particularly ALS. Using animal models, systemic delivery of human recombinant IGF-I (mature IGF-I) has been used in animal models and to treat ALS patients. Most recently, it was reported that exercise, when combined with IGF-I gene therapy by AAV2 vector, has some synergistic effects in treatment of an animal model of ALS [Kaspar et al., 2005].

However, the data presented here indicate it is the activity of MGF, not that of ordinary IGF-I, that will be most for use in the treatment of muscle wasting, because it offers an effective method of replenishing the muscle satellite (stem) cell pool that is required for muscle maintenance and repair. This supports the use of peptides of the invention as therapeutic agents for muscle degeneration in disorders such as CMD, FSHD and ALS in which there is an apparent impairment in expressing the MGF splice variant. There is also the potential for using peptides of the invention to multiply the muscle satellite cells in culture for cell therapy purposes.

7. Cell Proliferation Assays with 8 Amino Acid Peptides

7.1 DMGF and CMGF Peptides

The 8 amino acid peptides described in 1.3.1 above and referred to in FIG. 8A as DMGF and CMGF were tested for the ability to induce proliferation of C2C12 muscle cells at a density of 2000 cells per well in a medium containing DMEM (1000 mg/L glucose), BSA (10 ug/ml) and IGF-I (2 ng per ml). Concentrations of 2, 5, 50 and 100 ng/ml of DMGF and CMGF were tested (See the left-hand and middle sets of results in FIG. 8), along with 2, 5, 50 and 100 ng/ml IGF-I alone (See the right-hand set of results in FIG. 8). After 36 hours incubation, an Alamar Blue assay was used to assess the level of cell proliferation achieved. A control containing only the medium was also provided.

Both DMGF and CMGF induced cell proliferation. The results are shown in FIG. 8A in terms of fluorescence in the Alamar Blue Assay. All values for DMGF and CMGF, and those for IGF-I alone, were statistically different to the control value for the medium only. Increasing levels of proliferation were observed with increasing concentration of DMGF/CMGF.

7.2 Peptides A2, A4, A6 and A8

The 8 amino acid peptides described in 1.3.2 above and referred to in FIG. 8B as A2, A4, A6 and A8 were tested for the ability to induce proliferation of C2C12 muscle cells at a density of 500 cells per well. Cultivation was carried out for 24 hours in 10% FBS, followed by starvation for 24 hours in 0.1% BSA, stimulation for 24 hours and then treatment with BrdU for 5 hours. Concentrations of 0.1, 1, 10 and 100 ng/ml of peptides A2, A4, A6 and A8 were tested, along with 0.1, 1, 10 and 100 ng/ml IGF-I (See the right-hand set of results in FIG. 8B). Incorporation of BrdU was measured to assess the level of cell proliferation achieved. Controls containing no cells, medium only, 5% FBS and no BrdU were also provided.

Peptides A2, A4, A6 and A8 induced cell proliferation. The results are shown in FIG. 8 in terms of fluorescence (absorbence at 370 nm; mean plus standard error across 4 wells).

8. Cell Proliferation Assays with Human Primary Cells (HSMM)

The 24 amino acid peptide described in 1.4 above and referred to in FIGS. 9-11 as A5 was tested for the ability to induce proliferation of human muscle progenitor cells (Cambrex). These are commercially available primary human muscle cells, ie human muscle stem (progenitor) cells. They are also sometimes known as Human Skeletal Muscle Myoblasts (HSMM). Cells were obtained from a 39 year old male subject.

Cultivation was carried out for 24 hours in 200 μl of SkGM2 medium supplemented with hEGF, L-Glut, dexamethasone, antibiotics and 10% FCS. The cultivation medium was then removed and the cells were washed twice in serum free medium.

A5 was tested for the ability to induce proliferation of Cambrex HSMM at a density of 500 (FIGS. 9 and 10) or 1000 (FIG. 11) cells per well in Cambrex SkGM2 medium supplemented with hEGF, L-Glut, dexamethasone and antibiotics. Concentrations of 0.1, 1, 10, 100 and 500 ng/ml of A5 were tested (See the left-hand sets of results in FIGS. 9A, 10A and 11A), along with 0.1, 10 and 100 ng/ml IGF-I alone (See results in FIGS. 9A, 10A and 11A). Concentrations of 0.1, 1, 10, 100 and 500 ng/ml of A5 were also tested in the presence of 2 ng/ml IGF-I (See the left-hand set of results in FIGS. 9B, 10B and 11B). After 48 hours incubation, the cells were treated with BrdU for 5 hours. Incorporation of BrdU was measured to assess the level of cell proliferation achieved. Controls containing no cells, medium only, 5% FBS and no BrdU were also provided.

IGF-I alone had no significant effect on the proliferation of HSMM at any dose (see FIGS. 9-11). After 48 hours, the A5 peptide had a significant effect (P<0.1) on the proliferation of HSMM when used in isolation at doses of 10 ng/ml and below (FIGS. 9A and 10A). Addition of 2 ng/ml IGF-I to the medium in combination with A5 resulted in a significant effect on the proliferation of HSMM at a higher confidence level (P<0.001; FIGS. 9B, 10B and 11B). As the cells are comparatively slow growing, it is recommended to increase incubation period with peptide to 72 hours. Secondly, the signal would be enhanced by increasing the BrdU exposure time.

### REFERENCES

Domanska-Janik et al. Brain Res. Mol. Brain Res. 121, 50-59 (2004)
Hill and Goldspink, J. Physiol. 549.2, 409-418 (2003)
McKoy et al, J. Physiol. 516.2, 573-592 (1999)
Sarnowska, Folia Neuropathol. 40[2], 101-106 (2002)
Stoppini, et al, J. Neurosci Methods 37, 173-182 (1991)
Yang et al, J. Muscle Res. Cell Motil. 17, 487-495 (1996)
Yang and Goldspink, FEBS Letts. 522, 156-160 (2002)
Auluck et al, Euro. J. Oral Sci. 113: 218-244 (2005)
De Bari et al, J. Cell Biol. 60: 909-918 (2003)
Dluzniewska et al, FASEB J. 19: 1896-1898 (2005)
Fiske and Subbarow, J. Biol. Chem. 66: 375-400 (1925)
Goldspink et al, J. Physiol. 495P: 162-163P (1996).
Goto et al, Anal. Biochem. 272: 135-142 (1999)
Hameed et al, J. Physiol. 555: 231-240 (2004)
Kaspar et al, Ann. Neurol. 57: 649-655 (2005)
Lewis et al, Muscle Res. Cel. Motil. 21: 223-233 (2000)
Sinanan et al, Biotechnol. Appl. Biochem. 40:25-34 (2004)

1. A polypeptide comprising up to 50 amino acid residues;
said polypeptide comprising a sequence of amino acids derived from the C-terminal E peptide of a Mechano Growth Factor (MGF) isoform of Insulin-like Growth Factor I (IGF-I);
said polypeptide incorporating one or more modifications that give it increased stability compared to the unmodified MGF E peptide;
and said polypeptide possessing biological activity.

2. A polypeptide of claim 1 wherein said biological activity is selected from the ability to increase muscle strength, cardioprotective ability and neuroprotective ability.

3. A polypeptide of claim 1 wherein at least one of said modifications is to said sequence of amino acids that is derived from said C-terminal E peptide.

4. A polypeptide of claim 1 wherein said modifications include one or more conversions of an L-form amino acid to the corresponding D-form amino acid.

5. A polypeptide of claim 1 wherein said modifications include PEGylation or the addition of a hexanoic or amino-hexanoic acid moiety

6. A polypeptide of claim 5 wherein said PEGylation or addition of a hexanoic or amino-hexanoic acid moiety is at the N-terminal.

7. A polypeptide of claim 1 wherein said modifications include cyclisation of the polypeptide.

8. A polypeptide of claim 1 wherein said modifications include the substitution of one or more amino acids.

9. A polypeptide of claim 8 wherein said substitution includes the replacement with Alanine of an amino acid other than Alanine.

10. A polypeptide of claim 1 wherein said C-terminal E peptide is the Rat Eb peptide of SEQ ID NO: 13 or the Rabbit Eb peptide of SEQ ID NO: 14.

11. A polypeptide of claim 1 wherein said C-terminal E peptide is the human Ec peptide of SEQ ID NO: 27 or the peptide of SEQ ID NO: 15.

12. A polypeptide of claim 11 wherein the modifications include PEGylation or the addition of a hexanoic or amino-hexanoic acid moiety.

13. A polypeptide of claim 12 wherein said PEGylation or addition of a hexanoic or amino-hexanoic acid moiety is at the N-terminal.

14. A polypeptide of claim 11 wherein said modifications include one or more conversions of an L-form amino acid to the corresponding D-form amino acid.

15. A polypeptide of claim 14 wherein one or both of the Arginine residues at positions 14 and 15 of SEQ ID NO: 27 or 15 is in the D-form.

16. A polypeptide of claim 15 wherein both of the Arginine residues at positions 14 and 15 of SEQ ID NO: 27 or 15 are in the D-form.

17. A polypeptide of claim 11 wherein said modifications include the substitution of one or more amino acids.

18. A polypeptide of claim 17 wherein said substitution is at position 5, 12, 14 or 18.

19. A polypeptide of claim 18 wherein said substitution includes the replacement with Alanine of an amino acid other than Alanine.

20. A polypeptide of claim 19 wherein said Alanine substitution is one or more of (a) Serine to Alanine at position 5, (b) Serine to Alanine at position 12, (c) Arginine to Alanine at position 14 and (d) Serine to Alanine at position 18 of SEQ ID NO: 15 or 27.

21. A polypeptide of claim 1 wherein said C-terminal peptide is the polypeptide of SEQ ID NO: 33 or 34.

22. A polypeptide of claim 21 wherein the modifications include PEGylation or the addition of a hexanoic or amino-hexanoic acid moiety.

23. A polypeptide of claim 22 wherein said PEGylation or addition of a hexanoic or amino-hexanoic acid moiety is at the N-terminal.

24. A polypeptide of claim 20 wherein said modifications include the substitution of one or more amino acids.

25. A polypeptide of claim 24 wherein said substitution is at position 2.

26. A polypeptide of claim 25 wherein said substitution includes the replacement with Alanine of an amino acid other than Alanine.

27. A polypeptide of claim 26 wherein said Alanine substitution is one or more of (a) Serine to Alanine at position 2.

28. A polypeptide of claim 21 whose sequence is that of SEQ ID NO: 33, 34, 35 or 36.

29. A polypeptide of claim 1 wherein said modifications include the truncation by one or two amino acids of the C-terminus of said sequence of amino acids that is derived from said C-terminal E peptide.

30. A polypeptide of claim 29 whose sequence is that of the polypeptide of SEQ ID NO: 21.

31. A polypeptide of claim 11 whose sequence is that of the polypeptide of SEQ ID NO: 16, 17, 18, 19, 28, 29, 30 or 31.

32. A polypeptide of claim 11 whose sequence is that of SEQ ID NO: 15 or 27 but which is PEGylated at the N-terminus and wherein both of the Arginine residues at positions 14 and 15 of SEQ ID NO: 15 or 27 are in the D-form.

33. A polypeptide of claim 11 whose sequence is that of SEQ ID NO: 15 or 27, wherein both of the Arginine residues at positions 14 and 15 of SEQ ID NO: 15′ or 27 are in the D-form, and which is not PEGylated.

34. A polypeptide of claim 1 which is amidated at the C-terminus.

35. An extended polypeptide comprising a polypeptide of claim 1 extended by non-wild-type amino acid sequence N-terminal and/or C-terminal to said polypeptide of claim 1.

36. An extended polypeptide of claim 35, wherein said extension comprises a Cysteine residue at the C-terminus and/or a D-Arginine residue at the N-terminus.

37. A polypeptide of claim 1 whose stability, as measured by half-life in human plasma, is at least 10% greater than that of the unmodified E peptide.

38. A polypeptide of claim 37 whose stability, as measured by half-life in human plasma, is at least 50% greater than that of the unmodified E peptide.

39. A polypeptide of claim 38 whose stability, as measured by half-life in human plasma, is at least 100% or more greater than that of the unmodified E peptide.

40. A polypeptide of claim 1 whose half-life in human plasma is at least 2 hours.

41. A polypeptide of claim 40 whose half-life in human plasma is at least 12 hours or at least 24 hours.

42. A composition comprising a polypeptide of claim 1 and a carrier.

43. A composition comprising an extended polypeptide of claim 35 and a carrier.

44. A pharmaceutical composition comprising a polypeptide of claim 1 and a pharmaceutically acceptable carrier.

45. A method of treating a muscular disorder by administering to a patient in need thereof an effective amount of a polypeptide of claim 1.

46. A method of claim 45 wherein said muscular disorder is a disorder of skeletal muscle.

47. A method of claim 46 wherein said muscular disorder is muscular dystrophy or related progressive skeletal muscle weakness or wasting, muscle atrophy, cachexia, muscle weakness; sarcopenia or frailty in an elderly subject; or wherein said polypeptide or extended polypeptide is administered for the purpose of muscle repair following trauma.

48. A method of claim 47 wherein said muscular dystrophy is Duchenne or Becker muscular dystrophy, facioscapulohumeral muscular dystrophy (FSHD) or congenital muscular dystrophy (CMD); said muscle atrophy is disuse atrophy, glucocorticoid-induced atrophy, muscle atrophy in an ageing subject or muscle atrophy induced by spinal cord injury or neuromuscular disease; said cachexia is associated with, cancer, AIDS, Chronic Obstructive Pulmonary Disease (COPD), a chronic inflammatory disease or burns injury; or said muscle weakness is in the urinary sphincter, anal sphincter or pelvic floor muscles.

49. A method of claim 45 wherein said muscular disorder is a disorder of cardiac muscle.

50. A method of claim 49 wherein said polypeptide or extended polypeptide is administered for the purpose of prevention or limitation of myocardial damage in response to ischemia or mechanical overload of the heart; to promote cardiac muscle synthesis; to improve cardiac output by increasing heart stroke volume; to treat a cardiomyopathy; in response to an acute heart failure or acute insult to the heart; to treat pathological heart hypertrophy; or to treat congestive heart failure.

51. A method according to claim 50 wherein said acute heart failure or acute insult comprises myocarditis or myocardial infarction.

52. A method of treating a neurological disorder by administering to a patient in need thereof an effective amount of a polypeptide of claim 1.

53. A method of claim 52 wherein said polypeptide or extended polypeptide is administered for the purpose of prevention of neuronal loss associated with a disorder of, damage to, the nervous system, or for maintenance of the central nervous system (CNS).

54. A method of claim 53 wherein said neuronal loss is associated with a neurodegenerative disorder, nerve damage or ischemia.

55. A method according to claim 54 wherein said disorder is amyotrophic lateral sclerosis; spinal muscular atrophy; progressive spinal muscular atrophy; infantile or juvenile muscular atrophy, poliomyelitis or post-polio syndrome; a disorder caused by exposure to a toxin, motoneurone trauma, a motoneurone lesion or nerve damage; an injury that affects motoneurones; motoneurone loss associated with ageing; autosomal or sex-linked muscular dystrophy; Alzheimer's disease; Parkinson's disease; diabetic neuropathy; a peripheral neuropathy; an embolic or haemorrhagic stroke; alcohol-related brain damage; or wherein said polypeptide or extended polypeptide is administered for the purpose of nerve repair following trauma.

56. A method of treating a neurological disorder by administering to a patient in need thereof an effective amount of:
a polypeptide comprising up to 50 amino acid residues, said polypeptide comprising a sequence of amino acids derived from the C-terminal E peptide of a Mechano Growth Factor (MGF) isoform of Insulin-like Growth Factor I (IGF-I); or an extended polypeptide comprising said polypeptide and extended by non-wild-type amino acid sequence N-terminal and/or C-terminal to said polypeptide;
and said polypeptide or extended polypeptide possessing biological activity.

57. A method of claim 56 wherein said biological activity is neuroprotective ability.

58. A method of claim 56 wherein said polypeptide or extended polypeptide is administered for the purpose of prevention of neuronal loss associated with a disorder of, or damage to, the nervous system, or for maintenance of the central nervous system (CNS).

59. A method of claim 58 wherein said neuronal loss is associated with a neurodegenerative disorder, nerve damage or ischemia.

60. A method according to claim 56 wherein said disorder is amyotrophic lateral sclerosis; spinal muscular atrophy; progressive spinal muscular atrophy; infantile or juvenile muscular atrophy, poliomyelitis or post-polio syndrome; a disorder caused by exposure to a toxin, motoneurone trauma, a motoneurone lesion or nerve damage; an injury that affects motoneurones; motoneurone loss associated with ageing; autosomal or sex-linked muscular dystrophy; Alzheimer's disease; Parkinson's disease; diabetic neuropathy; a peripheral neuropathy; an embolic or haemorrhagic stroke; alcohol-related brain damage; or wherein said polypeptide or extended polypeptide is administered for the purpose of nerve repair following trauma.

61. A method of treating a disorder of cardiac muscle by administering to a patient in need thereof an effective amount of:
a polypeptide comprising up to 50 amino acid residues, said polypeptide comprising a sequence of amino acids derived from the C-terminal E peptide of a Mechano Growth Factor (MGF) isoform of Insulin-like Growth Factor I (IGF-I); or an extended polypeptide comprising said polypeptide and extended by non-wild-type amino acid sequence N-terminal and/or C-terminal to said polypeptide;
and said polypeptide possessing biological activity.

62. A method of claim 61 wherein said biological activity is cardioprotective ability.

63. A method according to claim 61 wherein said polypeptide or extended polypeptide is administered for the purpose of prevention or limitation of myocardial damage in response to ischemia or mechanical overload of the heart;
to promote cardiac muscle synthesis; to improve cardiac output by increasing heart stroke volume; to treat a cardiomyopathy; in response to an acute heart failure or acute insult to the heart; to treat pathological heart hypertrophy; or to treat congestive heart failure.

64. A method according to claim 63 wherein said acute heart failure or acute insult comprises myocarditis or myocardial infarction.

65. A method of claim 57 wherein said C-terminal E peptide is the Rat Eb peptide of SEQ ID NO: 13, the Rabbit Eb peptide of SEQ ID NO: 14, the human Ec peptide of SEQ ID NO: 27, the peptide of SEQ ID NO: 15 or the peptide of SEQ ID NO: 33 or 34.

66. A method of claim 61 wherein said C-terminal E peptide is the Rat Eb peptide of SEQ ID NO: 13, the Rabbit Eb peptide of SEQ ID NO: 14, the human Ec peptide of SEQ ID NO: 27, the peptide of SEQ ID NO: 15 or the peptide of SEQ ID NO: 33 or 34.

67. A method of claim 65 wherein said polypeptide or extended polypeptide comprises the sequence of SEQ ID NO: 13, 14, 15, 27, 33 or 34.

68. A method of claim 66 wherein said polypeptide or extended polypeptide comprises the sequence of SEQ ID NO: 13, 14, 15, 27, 33 or 34.

69. A method of claim 67 wherein the sequence of said polypeptide is that of the sequence of the sequence of SEQ ID NO: 13, 14, 15, 27, 33 or 34.

70. A polypeptide whose sequence is that of SEQ ID NO: 27, wherein one or both of the Arginine residues at positions 14 and 15 of SEQ ID NO: 27 is in the D-form.

71. A polypeptide of claim 70 wherein both of the Arginine residues at positions 14 and 15 of SEQ ID NO: 27 are in the D-form.

72. A polypeptide whose sequence is that of SEQ ID NO: 33, 34, 35 or 36.

73. A polypeptide of claim 70 further comprising one to five additional amino acids at the C-terminus and/or one to five additional amino acids the N-terminus.

74. A polypeptide of claim 73 wherein one or more of said additional amino acids is a D-form amino acid.

75. A polypeptide of claim 74 wherein one additional D-form amino acid is present at the N-terminus.

76. A polypeptide of claim 75 wherein said one additional D-form amino acid is D-Arginine.

77. A polypeptide of claim 76 wherein no additional amino acids are present at the C-terminus.

78. A polypeptide of claim 70 wherein one additional amino acid is present at the C-terminus and is Cysteine.

79. A polypeptide according to claim 78 wherein no additional amino acids are present at the N-terminus.

80. A polypeptide whose sequence is that of SEQ D NO: 15 or 27, plus one additional Cysteine residue at the C-terminus and optionally one to four further amino acids at the C-terminus and/or one to five further amino acids at the N-terminus.

81. A polypeptide of claim 80 wherein one or both of the Arginine residues at positions 14 and 15 of SEQ ID NO: 15 or 27 is in the D-form.

82. A polypeptide of claim 81 wherein both of the Arginine residues at positions 14 and 15 of SEQ ID NO: 27 or 15 are in the D-form.

83. A polypeptide of claim 80 wherein one or more of said further amino acids is a D-form amino acid.

84. A polypeptide of claim 83 wherein one D-form amino acid is present at the N-terminus.

85. A polypeptide of claim 84 wherein said one D-form amino acid is D-Arginine.

86. A polypeptide of claim 70 which is amidated at the C-terminus.

87. A polypeptide of claim 70 which is PEGylated, or to which is attached a hexanoic or amino-hexanoic acid moiety.

88. A polypeptide of claim 87 wherein said PEGylation or attachment of a hexanoic or amino-hexanoic acid moiety is at the N-terminus.

89. A polypeptide of claim 70 which is not PEGylated.

90. A polypeptide of claim 11 which is amidated at the C-terminus.

91. A polypeptide of claim 15 which is amidated at the C-terminus.

92. A polypeptide of claim 21 which is amidated at the C-terminus.

93. A polypeptide of claim 28 which is amidated at the C-terminus.

94. A polypeptide of claim 31 which is amidated at the C-terminus.

95. A polypeptide of claim 33 which is amidated at the C-terminus.

96. A polypeptide of claim 72 which is amidated at the C-terminus.

97. A polypeptide of claim 72 which is PEGylated, or to which is attached a hexanoic or amino-hexanoic acid moiety.

98. A polypeptide of claim 97 wherein said PEGylation or attachment of a hexanoic or amino-hexanoic acid moiety is at the N-terminus.

99. A polypeptide of claim 72 which is not PEGylated.

100. A method of claim 68 wherein the sequence of said polypeptide is that of the sequence of the sequence of SEQ ID NO: 13, 14, 15, 27, 33 or 34.


SEQUENCE LISTING (36 sequences)

SEQ ID NO: 1 - DNA, 517 bp, Homo sapiens
  CDS (1)..(330)
ggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaagtatcagcccccatctaccaacaagaacacgaagtctcagagaaggaaaggaagtacatttgaagaacgcaagtagagggagtgcaggaaacaagaactacaggatgtagaagacccttctgaggagtgaagaaggacaggccaccgcaggaccctttgctctgcacagttacctgtaaacattggaataccggccaaaaaataagtttgatcacatttcaaagatggcatttcccccaatgaaatacacaagtaaacat

SEQ ID NO: 2 - protein, 110 aa, Homo sapiens
GPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGSTFEERK

SEQ ID NO: 3 - DNA, 539 bp, Rattus sp.
  CDS (1)..(336)
ggaccagagaccctttgcggggctgagctggtggacgctcttcagttcgtgtgtggaccaaggggcttttacttcaacaagcccacagtctatggctccagcattcggagggcaccacagacgggcattgtggatgagtgttgcttccggagctgtgatctgaggaggctggagatgtactgtgtccgctgcaagcctacaaagtcagctcgttccatccgggcccagcgccacactgacatgcccaagactcagaagtcccagcccctatcgacacacaagaaaaggaagctgcaaaggagaaggaaaggaagtacacttgaagaacacaagtagaggaagtgcaggaaacaagacctacagaatgtaggaggagcctcccgaggaacagaaaatgccacgtcaccgcaagatcctttgctgcttgagcaacctgcaaaacatcggaacacctgccaaatatcaataatgagttcaatatcatttcagagatgggcatttccctcaatgaaatacacaagtaaacattcccggaattc

SEQ ID NO: 4 - protein, 111 aa, Rattus sp.
GPETLCGAELVDALQFVCGPRGFYFNKPTVYGSSIRRAPQTGIVDECCFRSCDLRRLEMYCVRCKPTKSARSIRAQRHTDMPKTQKSQPLSTHKKRKLQRRRKGSTLEEHK

SEQ ID NO: 5 - DNA, 523 bp, Oryctolagus cuniculus
  CDS (1)..(336)
ggaccggagacgctctgcggtgctgagctggtggatgctcttcagttcgtgtgtggagacaggggcttttatttcaacaagcccacaggatacggctccagcagtcggagggcacctcagacaggcatcgtggatgagtgctgcttccggagctgtgatctgaggaggctggagatgtactgtgcacccctcaagccggcaaaggcagcccgctccgtccgtgcccagcgccacaccgacatgcccaagactcagaagtatcagcctccatctaccaacaagaaaatgaagtctcagaggagaaggaaaggaagtacatttgaagaacacaagtagagggagtgcaggaaacaagaactacaggatgtaggaagacccttctgaggagtgaagaaggacaggccaccgcaggaccctttgctctgcacagttacctgtaaacattggaataccggccaaaaaataagtttgatcacatttcaaagatggcatttcccccaatgaaatacacaagtaaacattc

SEQ ID NO: 6 - protein, 111 aa, Oryctolagus cuniculus
GPETLCGAELVDALQFVCGDRGFYFNKPTGYGSSSRRAPQTGIVDECCFRSCDLRRLEMYCAPLKPAKAARSVRAQRHTDMPKTQKYQPPSTNKKMKSQRRRKGSTFEEHK

SEQ ID NO: 7 - DNA, 317 bp, Homo sapiens
  CDS (1)..(315)
ggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgag

SEQ ID NO: 8 - protein, 105 aa, Homo sapiens
GPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 9 - DNA, 487 bp, Rattus sp.
  CDS (1)..(318)
ggaccagagaccctttgcggggctgagctggtggacgctcttcagttcgtgtgtggaccaaggggcttttacttcaacaagcccacagtctatggctccagcattcggagggcaccacagacgggcattgtggatgagtgttgcttccggagctgtgatctgaggaggctggagatgtactgtgtccgctgcaagcctacaaagtcagctcgttccatccgggcccagcgccacactgacatgcccaagactcagaaggaagtacacttgaagaacacaagtagaggaagtgcaggaaacaagacctacagaatgtaggaggagcctcccgaggaacagaaaatgccacgtcaccgcaagatcctttgctgcttgagcaacctgcaaaacatcggaacacctgccaaatatcaataatgagttcaatatcatttcagagatgggcatttccctcaatgaaatacacaagtaaacattcccggaattc

SEQ ID NO: 10 - protein, 105 aa, Rattus sp.
GPETLCGAELVDALQFVCGPRGFYFNKPTVYGSSIRRAPQTGIVDECCFRSCDLRRLEMYCVRCKPTKSARSIRAQRHTDMPKTQKEVHLKNTSRGSAGNKTYRM

SEQ ID NO: 11 - DNA, 471 bp, Oryctolagus cuniculus
  CDS (1)..(318)
ggaccggagacgctctgcggtgctgagctggtggatgctcttcagttcgtgtgtggagacaggggcttttatttcaacaagcccacaggatacggctccagcagtcggagggcacctcagacaggcatcgtggatgagtgctgcttccggagctgtgatctgaggaggctggagatgtactgtgcacccctcaagccggcaaaggcagcccgctccgtccgtgcccagcgccacaccgacatgcccaagactcagaaggaagtacatttgaagaacacaagtagagggagtgcaggaaacaagaactacaggatgtaggaagacccttctgaggagtgaagaaggacaggccaccgcaggaccctttgctctgcacagttacctgtaaacattggaataccggccaaaaaataagtttgatcacatttcaaagatggcatttcccccaatgaaatacacaagtaaacattc

SEQ ID NO: 12 - protein, 105 aa, Oryctolagus cuniculus
GPETLCGAELVDALQFVCGDRGFYFNKPTGYGSSSRRAPQTGIVDECCFRSCDLRRLEMYCAPLKPAKAARSVRAQRHTDMPKTQKEVHLKNTSRGSAGNKNYRM

SEQ ID NO: 13 - protein, 25 aa, Artificial Sequence
  Synthetic peptide
SQPLSTHKKRKLQRRRKGSTLEEHK

SEQ ID NO: 14 - protein, 25 aa, Artificial Sequence
  Synthetic peptide
YQPPSTNKKMKSQRRRKGSTFEEHK

SEQ ID NO: 15 - protein, 24 aa, Artificial sequence
  Synthetic peptide
YQPPSTNKNTKSQRRKGSTFEEHK

SEQ ID NO: 16 - protein, 24 aa, Artificial sequence
  Synthetic peptide
YQPPATNKNTKSQRRKGSTFEEHK

SEQ ID NO: 17 - protein, 24 aa, Artificial sequence
  Synthetic peptide
YQPPSTNKNTKAQRRKGSTFEEHK

SEQ ID NO: 18 - protein, 24 aa, Artificial sequence
  Synthetic peptide
YQPPSTNKNTKSQRRKGATFEEHK

SEQ ID NO: 19 - protein, 24 aa, Artificial sequence
  Synthetic peptide
YQPPSTNKNTKSQARKGSTFEEHK

SEQ ID NO: 20 - protein, 24 aa, Artificial sequence
  Synthetic peptide
YQPPSTNKNTKSQAAKGSTFEEHK

SEQ ID NO: 21 - protein, 22 aa, Artificial sequence
  Synthetic peptide
YQPPSTNKNTKSQARKGSTFEE

SEQ ID NO: 22 - protein, 20 aa, Artificial sequence
  Synthetic peptide
YQPPSTNKNTKSQARKGSTF

SEQ ID NO: 23 - protein, 21 aa, Artificial sequence
  Synthetic peptide
PSTNKNTKSQARKGSTFEEHK

SEQ ID NO: 24 - protein, 19 aa, Artificial sequence
  Synthetic peptide
TNKNTKSQARKGSTFEEHK

SEQ ID NO: 25 - DNA, 517 bp, Homo sapiens
  CDS (1)..(330)
ggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaagtatcagcccccatctaccaacaagaacacgaagtctcagagaaggaaaggaagtacatttgaagaacacaagtagagggagtgcaggaaacaagaactacaggatgtagaagacccttctgaggagtgaagaaggacaggccaccgcaggaccctttgctctgcacagttacctgtaaacattggaataccggccaaaaaataagtttgatcacatttcaaagatggcatttcccccaatgaaatacacaagtaaacat

SEQ ID NO: 26 - protein, 110 aa, Homo sapiens
GPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGSTFEEHK

SEQ ID NO: 27 - protein, 24 aa, Artificial sequence
  Synthetic peptide
YQPPSTNKNTKSQRRKGSTFEERK

SEQ ID NO: 28 - protein, 24 aa, Artificial sequence
  Synthetic peptide
YQPPATNKNTKSQRRKGSTFEERK

SEQ ID NO: 29 - protein, 24 aa, Artificial sequence
  Synthetic peptide
YQPPSTNKNTKAQRRKGSTFEERK

SEQ ID NO: 30 - protein, 24 aa, Artificial sequence
  Synthetic peptide
YQPPSTNKNTKSQRRKGATFEERK

SEQ ID NO: 31 - protein, 24 aa, Artificial sequence
  Synthetic peptide
YQPPSTNKNTKSQARKGSTFEERK

SEQ ID NO: 32 - protein, 24 aa, Artificial sequence
  Synthetic peptide
YQPPSTNKNTKSQAAKGSTFEERK

SEQ ID NO: 33 - protein, 8 aa, Artificial sequence
  Synthetic peptide
GSTFEEHK

SEQ ID NO: 34 - protein, 8 aa, Artificial sequence
  Synthetic peptide
GSTFEERK

SEQ ID NO: 35 - protein, 8 aa, Artificial sequence
  Synthetic peptide
GATFEEHK

SEQ ID NO: 36 - protein, 8 aa, Artificial sequence
  Synthetic peptide
GATFEERK